SEQUENCE LISTING (53 sequences)

SEQ ID NO: 1 - protein, 10 aa, artificial
  1022C3 antibody CDR-H1 (X1)
  VARIANT (5)..(5): X is N, D, Q, S, E, R, K, H, W, Y or T
GFSLXTSGMG

SEQ ID NO: 2 - protein, 7 aa, artificial
  1022C3 antibody CDR-H2
IYWDDDK

SEQ ID NO: 3 - protein, 15 aa, artificial
  1022C3 antibody CDR-H3
ARRSHFGNYYYAMDY

SEQ ID NO: 4 - protein, 6 aa, artificial
  1022C3 antibody CDR-L1
QDISSY

SEQ ID NO: 5 - protein, 3 aa, artificial
  1022C3 antibody CDR-L2
YTS

SEQ ID NO: 6 - protein, 9 aa, artificial
  1022C3 antibody CDR-L3
QQGKTLPLT

SEQ ID NO: 7 - protein, 10 aa, artificial
  1022C3 antibody CDR-H1 (N)
GFSLNTSGMG

SEQ ID NO: 8 - protein, 10 aa, artificial
  1022C3 antibody CDR-H1 (D)
GFSLDTSGMG

SEQ ID NO: 9 - protein, 123 aa, artificial
  1022C3 antibody heavy chain variable domain (X1)
  VARIANT (30)..(30): X is N, D, Q, S, E, R, K, H, W, Y or T
QVTLKESGPGILQPSQTLSLTCSFSGFSLXTSGMGVSWIRQPSGKGLEWLAHIYWDDDKRYNPSLKSRLTISKDTSSNQVFLKITSLDTADTATYYCARRSHFGNYYYAMDYWGQGTSVTVTS

SEQ ID NO: 10 - protein, 107 aa, artificial
  1022C3 antibody light chain variable domain
DVQMTQTTSSLSASLGDRVTISCRASQDISSYLNWYQQKPDGTFKLLIFYTSRLQSGVPSKYSGSGSGTDYSLTISNLEQEDIAAYFCQQGKTLPLTFGAGTKLELK

SEQ ID NO: 11 - protein, 123 aa, artificial
  1022C3 antibody heavy chain variable domain (N)
QVTLKESGPGILQPSQTLSLTCSFSGFSLNTSGMGVSWIRQPSGKGLEWLAHIYWDDDKRYNPSLKSRLTISKDTSSNQVFLKITSLDTADTATYYCARRSHFGNYYYAMDYWGQGTSVTVTS

SEQ ID NO: 12 - protein, 123 aa, artificial
  1022C3 antibody heavy chain variable domain (D)
QVTLKESGPGILQPSQTLSLTCSFSGFSLDTSGMGVSWIRQPSGKGLEWLAHIYWDDDKRYNPSLKSRLTISKDTSSNQVFLKITSLDTADTATYYCARRSHFGNYYYAMDYWGQGTSVTVTS

SEQ ID NO: 13 - DNA, 30 bp, artificial
  1022C3 antibody CDR-H1 (X2)
  misc_feature (13)..(15): nnn is AAC, AAT, GAC, GAT, CAG, CAA, TCG, TCT, TCA, TCC, AGT, AGC, GAA, GAG, CGA, CGT, CGC, CGG, AGA, AGG, AAA, AAG, CAT, CAC, TGG, TAT, TAC, ACT, ACC, ACG or ACA
gggttttcactgnnnacttctggtatgggt

SEQ ID NO: 14 - DNA, 21 bp, artificial
  1022C3 antibody CDR-H2
atttactgggatgacgacaag

SEQ ID NO: 15 - DNA, 45 bp, artificial
  1022C3 antibody CDR-H3
gctcgaagatctcactttggtaactattactatgctatggactac

SEQ ID NO: 16 - DNA, 18 bp, artificial
  1022C3 antibody CDR-L1
caggacattagtagttat

SEQ ID NO: 17 - DNA, 9 bp, artificial
  1022C3 antibody CDR-L2
tacacatca

SEQ ID NO: 18 - DNA, 27 bp, artificial
  1022C3 antibody CDR-L3
caacagggtaaaacacttccgctcacg

SEQ ID NO: 19 - DNA, 30 bp, artificial
  1022C3 antibody CDR-H1 (aac)
gggttttcactgaacacttctggtatgggt

SEQ ID NO: 20 - DNA, 30 bp, artificial
  1022C3 antibody CDR-H1 (aat)
gggttttcactgaatacttctggtatgggt

SEQ ID NO: 21 - DNA, 30 bp, artificial
  1022C3 antibody CDR-H1 (gac)
gggttttcactggacacttctggtatgggt

SEQ ID NO: 22 - DNA, 30 bp, artificial
  1022C3 antibody CDR-H1 (gat)
gggttttcactggatacttctggtatgggt

SEQ ID NO: 23 - DNA, 369 bp, artificial
  1022C3 antibody heavy chain variable domain (X2)
  misc_feature (88)..(90): nnn is AAC, AAT, GAC, GAT, CAG, CAA, TCG, TCT, TCA, TCC, AGT, AGC, GAA, GAG, CGA, CGT, CGC, CGG, AGA, AGG, AAA, AAG, CAT, CAC, TGG, TAT, TAC, ACT, ACC, ACG or ACA
caggttactctgaaagagtctggccctgggatattgcagccctcccagaccctcagtctgacttgttctttctctgggttttcactgnnnacttctggtatgggtgtgagctggattcgtcagccttcaggaaagggtctggagtggctggcacacatttactgggatgacgacaagcgctataatccatccctgaagagccggctcacaatctccaaggatacctccagcaaccaggttttcctcaagatcaccagtttggacactgcagatactgccacatactactgtgctcgaagatctcactttggtaactattactatgctatggactactggggtcaaggaacctcagtcaccgtcacctca

SEQ ID NO: 24 - DNA, 369 bp, artificial
  1022C3 antibody heavy chain variable domain (aac)
caggttactctgaaagagtctggccctgggatattgcagccctcccagaccctcagtctgacttgttctttctctgggttttcactgaacacttctggtatgggtgtgagctggattcgtcagccttcaggaaagggtctggagtggctggcacacatttactgggatgacgacaagcgctataatccatccctgaagagccggctcacaatctccaaggatacctccagcaaccaggttttcctcaagatcaccagtttggacactgcagatactgccacatactactgtgctcgaagatctcactttggtaactattactatgctatggactactggggtcaaggaacctcagtcaccgtcacctca

SEQ ID NO: 25 - DNA, 369 bp, artificial
  1022C3 antibody heavy chain variable domain (gat)
caggttactctgaaagagtctggccctgggatattgcagccctcccagaccctcagtctgacttgttctttctctgggttttcactggatacttctggtatgggtgtgagctggattcgtcagccttcaggaaagggtctggagtggctggcacacatttactgggatgacgacaagcgctataatccatccctgaagagccggctcacaatctccaaggatacctccagcaaccaggttttcctcaagatcaccagtttggacactgcagatactgccacatactactgtgctcgaagatctcactttggtaactattactatgctatggactactggggtcaaggaacctcagtcaccgtcacctca

SEQ ID NO: 26 - DNA, 321 bp, artificial
  1022C3 antibody light chain variable domain
gatgtccagatgacacagactacatcctccctgtctgcctctctgggagacagagtcaccatcagttgcagggcaagtcaggacattagtagttatttaaattggtatcagcagaaaccagatggaacttttaaactcctgatcttctacacatcaagattacagtcaggagtcccatcaaaatacagtggcagtgggtctggaacagattattctctcaccattagtaacctggagcaagaagatattgccgcttacttttgccaacagggtaaaacacttccgctcacgttcggggctgggaccaagctggaactgaaa

SEQ ID NO: 27 - DNA, 369 bp, artificial
  1022C3 antibody heavy chain variable domain (aat)
caggttactctgaaagagtctggccctgggatattgcagccctcccagaccctcagtctgacttgttctttctctgggttttcactgaatacttctggtatgggtgtgagctggattcgtcagccttcaggaaagggtctggagtggctggcacacatttactgggatgacgacaagcgctataatccatccctgaagagccggctcacaatctccaaggatacctccagcaaccaggttttcctcaagatcaccagtttggacactgcagatactgccacatactactgtgctcgaagatctcactttggtaactattactatgctatggactactggggtcaaggaacctcagtcaccgtcacctca

SEQ ID NO: 28 - DNA, 369 bp, artificial
  1022C3 antibody heavy chain variable domain (gac)
caggttactctgaaagagtctggccctgggatattgcagccctcccagaccctcagtctgacttgttctttctctgggttttcactggacacttctggtatgggtgtgagctggattcgtcagccttcaggaaagggtctggagtggctggcacacatttactgggatgacgacaagcgctataatccatccctgaagagccggctcacaatctccaaggatacctccagcaaccaggttttcctcaagatcaccagtttggacactgcagatactgccacatactactgtgctcgaagatctcactttggtaactattactatgctatggactactggggtcaaggaacctcagtcaccgtcacctca

SEQ ID NO: 29 - protein, 824 aa, artificial
  ADAM17
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 30 - protein, 260 aa, artificial
  Metalloprotease domain of ADAM17
RADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERS

SEQ ID NO: 31 - protein, 89 aa, artificial
  Disintegrin domain of ADAM17
NKVCGNSRVDEGEECDPGIMYLNNDTCCNSDCTLKEGVQCSDRNSPCCKNCQFETAQKKCQEAINATCKGVSYCTGNSSECPPPGNAED

SEQ ID NO: 32 - protein, 108 aa, artificial
  Membrane proximal domain of ADAM17
DTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADN

SEQ ID NO: 33 - protein, 452 aa, artificial
  1022C3 antibody heavy chain Chimeric (c) IgG1
QVTLKESGPGILQPSQTLSLTCSFSGFSLNTSGMGVSWIRQPSGKGLEWLAHIYWDDDKRYNPSLKSRLTISKDTSSNQVFLKITSLDTADTATYYCARRSHFGNYYYAMDYWGQGTSVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 452 aa, artificial
  1022C3 antibody heavy chain Chimeric (c) IgG1 AlaAla
QVTLKESGPGILQPSQTLSLTCSFSGFSLNTSGMGVSWIRQPSGKGLEWLAHIYWDDDKRYNPSLKSRLTISKDTSSNQVFLKITSLDTADTATYYCARRSHFGNYYYAMDYWGQGTSVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 214 aa, artificial
  1022C3 antibody light chain Chimeric (c) IgG1
DVQMTQTTSSLSASLGDRVTISCRASQDISSYLNWYQQKPDGTFKLLIFYTSRLQSGVPSKYSGSGSGTDYSLTISNLEQEDIAAYFCQQGKTLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 1356 bp, artificial
  1022C3 antibody heavy chain Chimeric (c)
caggtgaccctgaaggaaagcggacctggcattctgcagccatcacaaacactttcactgacatgcagtttttccggtttttctctgaataccagcgggatgggggtcagctggatccgccagcccagcggtaagggactcgagtggcttgcccacatctattgggatgacgataagcggtacaacccctccctcaaaagcagactgactattagtaaagacacttctagtaatcaggtgttcttgaagatcacctctcttgacacagccgatactgcaacttactactgcgctaggcgatcccatttcggcaactattactatgccatggattattggggccagggcacatctgtcacagttacctctgcttccaccaagggcccttccgtgttcccgctagcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 37 - DNA, 1356 bp, artificial
  1022C3 antibody heavy chain Chimeric (c) AlaAla
caggtgaccctgaaggaaagcggacctggcattctgcagccatcacaaacactttcactgacatgcagtttttccggtttttctctgaataccagcgggatgggggtcagctggatccgccagcccagcggtaagggactcgagtggcttgcccacatctattgggatgacgataagcggtacaacccctccctcaaaagcagactgactattagtaaagacacttctagtaatcaggtgttcttgaagatcacctctcttgacacagccgatactgcaacttactactgcgctaggcgatcccatttcggcaactattactatgccatggattattggggccagggcacatctgtcacagttacctctgcttccaccaagggcccaagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgaggcggcgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggc

SEQ ID NO: 38 - DNA, 642 bp, artificial
  1022C3 antibody light chain Chimeric (c)
gacgtccaaatgacacagacaacttcttccctgagcgcttccttgggtgatcgagtgaccattagttgcagagcaagccaggacatctcttcatacctcaactggtatcagcaaaaacctgatggcaccttcaagctgcttattttctacaccagtcggttgcagtctggcgttccatcaaagtattccggctctgggagtggcaccgactactccctcaccatcagcaatcttgagcaagaggatatcgcagcctatttctgccagcagggaaagacactccccctgactttcggggctgggaccaagctggaactgaaacgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 39 - protein, 123 aa, artificial; identical to SEQ ID NO: 12
  1022C3 antibody heavy chain humanized (hz) consensus sequence
  VARIANT (3)..(3): Thr may be replaced by Gln
  VARIANT (5)..(5): Lys may be replaced by Gln
  VARIANT (11)..(11): Ile may be replaced by Leu
  VARIANT (12)..(12): Leu may be replaced by Val
  VARIANT (13)..(13): Gln may be replaced by Lys
  VARIANT (16)..(16): Gln may be replaced by Glu
  VARIANT (23)..(23): Ser may be replaced by Thr
  VARIANT (24)..(24): Phe may be replaced by Val
  VARIANT (36)..(36): Val may be replaced by Trp
  VARIANT (43)..(43): Ser may be replaced by Ala
  VARIANT (50)..(50): Leu may be replaced by Ile
  VARIANT (51)..(51): Ala may be replaced by Gly
  VARIANT (52)..(52): His may be replaced by Arg
  VARIANT (60)..(60): Arg may be replaced by Asn
  VARIANT (69)..(69): Leu may be replaced by Val
  VARIANT (71)..(71): Ile may be replaced by Met
  VARIANT (73)..(73): Lys may be replaced by Val
  VARIANT (77)..(77): Ser may be replaced by Lys
  VARIANT (80)..(80): Val may be replaced by Phe
  VARIANT (81)..(81): Phe may be replaced by Ser
  VARIANT (84)..(84): Ile may be replaced by Leu
  VARIANT (85)..(85): Thr may be replaced by Ser
  VARIANT (87)..(87): Leu may be replaced by Val
  VARIANT (88)..(88): Asp may be replaced by Thr
  VARIANT (89)..(89): Thr may be replaced by Ala
  VARIANT (94)..(94): Thr may be replaced by Val
  VARIANT (118)..(118): Ser may be replaced by Thr
  VARIANT (122)..(122): Thr may be replaced by Ser

SEQ ID NO: 40 - protein, 123 aa, artificial
  1022C3 antibody heavy chain Hz variable domain
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSS

SEQ ID NO: 41 - protein, 452 aa, artificial
  1022C3 antibody heavy chain Hz full IgG1
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 448 aa, artificial
  1022C3 antibody heavy chain Hz full IgG2
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 43 - protein, 469 aa, artificial
  1022C3 antibody heavy chain Hz full IgG3
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 44 - protein, 449 aa, artificial
  1022C3 antibody heavy chain Hz full IgG4
QVQLQESGPGLVKPSETLSLTCTVSGFSLDTSGMGVSWIRQPAGKGLEWIGHIYWDDDKRYNPSLKSRVTMSKDTSSNQVSLKLSSVTAADTAVYYCARRSHFGNYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 107 aa, artificial; identical to SEQ ID NO: 10
  1022C3 antibody light chain humanized (hz) consensus sequence
  VARIANT (2)..(2): Val may be replaced by Ile
  VARIANT (7)..(7): Thr may be replaced by Ser
  VARIANT (8)..(8): Thr may be replaced by Pro
  VARIANT (15)..(15): Leu may be replaced by Val
  VARIANT (22)..(22): Ser may be replaced by Thr
  VARIANT (41)..(41): Asp may be replaced by Gly
  VARIANT (42)..(42): Gly may be replaced by Lys
  VARIANT (43)..(43): Thr may be replaced by Ala
  VARIANT (44)..(44): Phe may be replaced by Pro
  VARIANT (49)..(49): Phe may be replaced by Tyr
  VARIANT (53)..(53): Arg may be replaced by Ser
  VARIANT (61)..(61): Lys may be replaced by Arg
  VARIANT (62)..(62): Tyr may be replaced by Phe
  VARIANT (71)..(71): Tyr may be replaced by Phe
  VARIANT (72)..(72): Ser may be replaced by Thr
  VARIANT (77)..(77): Asn may be replaced by Ser
  VARIANT (79)..(79): Glu may be replaced by Gln
  VARIANT (80)..(80): Gln may be replaced by Pro
  VARIANT (83)..(83): Ile may be replaced by Phe
  VARIANT (85)..(85): Ala may be replaced by Thr
  VARIANT (87)..(87): Phe may be replaced by Tyr
  VARIANT (100)..(100): Ala may be replaced by Gly
  VARIANT (104)..(104): Leu may be replaced by Val
  VARIANT (106)..(106): Leu may be replaced by Ile

SEQ ID NO: 46 - protein, 107 aa, artificial
  1022C3 antibody light chain Hz variable domain
DIQMTQSPSSLSASVGDRVTITCRASQDISSYLNWYQQKPGKAPKLLIFYTSRLQSGVPSRFSGSGSGTDFTLTISSLQPEDFATYYCQQGKTLPLTFGGGTKVEIK

SEQ ID NO: 47 - protein, 214 aa, artificial
  1022C3 antibody light chain Hz full
DIQMTQSPSSLSASVGDRVTITCRASQDISSYLNWYQQKPGKAPKLLIFYTSRLQSGVPSRFSGSGSGTDFTLTISSLQPEDFATYYCQQGKTLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 369 bp, artificial
  1022C3 antibody heavy chain Hz variable domain
caagttcagttgcaggaatccggtcctggtcttgttaagccttccgagactttgtccctcacttgtaccgtgtccggctttagccttgacactagcggaatgggagtgagttggatcaggcagcccgcaggaaaaggcctggagtggattggccatatatactgggatgatgataaaaggtacaaccccagcctgaagtcacgtgtgacaatgagcaaagacacatctagcaatcaggtgagtctgaaactgtcttctgtgaccgccgctgacaccgctgtgtactactgcgcccggcgctctcattttgggaattattattatgccatggactattgggggcagggcacaaccgtcaccgtgtcatca

SEQ ID NO: 49 - DNA, 321 bp, artificial
  1022C3 antibody light chain Hz variable domain
gacatccagatgacccaatccccatcatccttgagcgcttcagtgggagatagggtaaccatcacctgcagggcatctcaggatatcagctcttacctgaactggtaccagcagaagcccggaaaggcccctaaattgctgattttctacaccagccgcctgcagagtggagttcctagtcggttttccggcagtggctctgggactgatttcactctgactattagctctctgcagcccgaagacttcgccacatattattgccagcaggggaagacactccccctgacatttggcgggggcacaaaagtggagataaag

SEQ ID NO: 50 - protein, 97 aa, artificial
  Germline IGHV4-4*07
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCAR

SEQ ID NO: 51 - protein, 95 aa, artificial
  Germline IGKV1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 52 - protein, 20 aa, artificial
  Germline IGHJ6*01
YYYYYGMDVWGQGTTVTVSS

SEQ ID NO: 53 - protein, 12 aa, artificial
  Germline IGKJ4*01
LTFGGGTKVEIK

(19) United States
(12) Patent Application Publication
(10) Pub. No.: US 2019/0071518 A1
(43) Pub. Date: Mar. 7, 2019

(54) PHARMACEUTICAL COMPOSITION FOR THE TREATMENT OF ADAM17 SUBSTRATE DEPENDENT CANCERS

(71) Applicant: PIERRE FABRE MEDICAMENT, BOULOGNE-BILLANCOURT (FR)

(72) Inventors: Peter LOWE, CHAZEY BONS (FR); Nicolas BOUTE, CERNEX (FR); Alexandra JOUHANNEAUD, BONNEVILLE (FR)

(21) Appl. No.: 15/538,497

(22) PCT Filed: Jan. 4, 2016
(86) PCT No.: PCT/EP2016/050028

§ 371 Date: Jun. 21, 2017

(30) Foreign Application Priority Data
Dec. 24, 2014 (EP) 14307190.0

(51) Int. Cl. C07K 16/40; A61P 35/00; C07K 16/28

(52) U.S. Cl. CPC C07K 16/40; A61P 35/00; C07K 16/2896; C07K 2317/24; C07K 2317/92; C07K 2317/41; C07K 2317/565; C07K 2317/76; C07K 2317/30

(57) ABSTRACT

The present disclosure relates to the treatment of cancers and, more particularly, to the treatment of ADAM17 substrate dependant cancers which are refractory. The pharmaceutical composition contains an ADAM17 antibody (it recognizes an epitope within the membrane proximal domain of ADAM17 localized between the residues 564 and 642) characterized by the sequences of its variable chains.

The present invention relates to the treatment of cancers and, more particularly, to the treatment of ADAM17 substrate dependant cancers which are refractory or resistant for example to ErbB therapies.

Cancer treatment has advanced gradually with periodic major advances through the addition of novel approaches and targets. Surgery, followed by hormone and then radiation therapy marked pivotal advances in the 19th and early 20th centuries. The next great advance was the development of chemotherapies following World War II. The earliest immunotherapies followed with the development of cytokine and related therapeutics. The molecular biological revolution in the 1970s and 80s culminated with the sequencing of the human genome and the birth of targeted therapies. With a greater understanding of the signalling pathways employed in normal cells and their deregulation in cancer has permitted the development of novel medicines specifically targeting the deregulated elements.

In particular the ErbB family of receptors, typically involved in development, regeneration and homeostasis of tissues, were found to be frequently deregulated in tumours. ErbB1 more commonly known as epidermal growth factor receptor (EGFR) and its sister receptor ErbB2 more frequently known as Human epidermal growth factor receptor 2 (Her2) are the most studied of the family and targeted therapies have been generated to both. Monoclonal antibodies Cetuximab and Panitumumab targeting EGFR and Trastuzumab and Pertuzumab targeting Her2 are widely used in the treatment of cancer. Small molecule inhibitors of the tyrosine kinase activity of EGFR include Gefitinib, Erlotinib, and Lapatinib (that also inhibits Her2). Silencing these growth factor receptors demonstrates potent inhibition of cellular replication in vitro.

Despite the expected efficacy of the targeted therapies that have been developed, their real world success has been more measured. In the case of metastatic colorectal cancer (mCRC) for which Cetuximab is an approved therapy, the treatment is effective in only 20% of patients, and of those at least 75% will go on to develop a resistance to the therapy, leading to progressive disease. The question remains why only certain patients benefit from EGFR targeting therapies and why in turn resistance will almost inevitably develop.

To address this question one must consider that growth factor receptor signalling in the case of EGFR is ligand dependent. Once ligand is engaged, structural changes take place in the receptor and dimerisation occurs with a second EGFR leading to signal transduction. It has more recently been proposed that ligand engagement can also provoke heterodimersiation leading to EGFR associating with an alternative receptor to fulfil signal transduction. The heterodimeric partners may include Her2, Her3, cMET, Axl or as yet undescribed receptors that facilitate ligand induced signalling. Such heterodimeric ligand dependent signalling being resistant to EGFR targeting therapies and resulting in aberrant pro tumorigenic signals (Wheeler D L et al., Oncogene. 2008 Jun. 26; 27(28):3944-56).

When considering clinical data obtained in the evaluation of mCRC patients treated with Cetuximab, it has been observed that immediately following administration and throughout the course of the treatment period, EGFR ligand levels are elevated as a result of the treatment (Tabernero et al., J Clin Oncol., 2010 Mar. 1; 28(7):1181-9). In particular, Amphiregulin (AREG) and transforming growth factor alpha (TGFα) levels in serum samples were elevated during the administration of Cetuximab. The same phenomenon was observed in a cohort of mCRC patients treated with Cetuximab and irinotecan (Loupakis et al., Target Oncol., 2014 September; 9(3):205-14). In this study AREG and TGFα levels were elevated from baseline one hour after administering Cetuximab and levels were even higher fifty seven days after treatment began. Most interestingly patients in the Cetuximab monotherapy (Tabernero et al., 2010) study that showed a response to treatment in the six week evaluation period were those in whom the AREG and TGFα levels increased the least and represented twenty eight percent of the study population. It may therefore be considered that the abundance and increase of ligand is at least in part responsible for the inactivity of EGFR targeting therapies like Cetuximab. As therapies such as Cetuximab prevent ligand interaction with EGFR, it is logical that systemically circulating or tumour produced ligand levels will naturally increase in the presence of Cetuximab or similar treatments due to a lack of receptor for them to bind. With time unbound EGFR will once again be presented at the cell surface, however, free ligand levels are now elevated and will immediately stimulate signalling. As tumours posses greatly elevated levels of EGFR expression these cells will be the first to represent receptor and be newly stimulated into growth. It has also recently been postulated that quiescent cells within the heterogeneous cancer cell population are major sources of EGFR ligands and that EGFR targeting induces even greater levels of expression (Hobor. S et al., Clin Cancer Res., 2014 Dec. 15; 20(24):6429-3).

The ligand dependent phenomenon is not limited to mCRC and has been reported from clinical investigations of multiple tumour types. Elevated levels of AREG and HB-EGF have been associated with poor outcomes and recurrent disease for patients suffering from squamous cell carcinomas of the head and neck (SCCHN). AREG in SCCHN patients was prognostic for outcome when treated with Cetuximab in combination with docetaxel as determined by AREG levels by immunohistochemical marking of tumour tissue (Tinhofer et al., Clin Cancer Res., 2011 Aug. 1; 17(15):5197-204). In a separate study, SCCHN cell lines that developed resistance to Cetuximab demonstrated elevated levels of HB-EGF and AREG, treatment of resistant cells resulted in elevation of TGFα levels (Hatakeyama et al; PLoS One., 2010 Sep. 13; 5(9):e12702). In the same study HB-EGF levels were evaluated in SCCHN patients that had recurrent disease and these levels were seen to be on average five times higher than in non recurrent patients (HB-EGF levels: Recurrent, 95 pg/ml, non recurrent, 23 pg/ml).

Resistance to therapy mediated by EGFR ligand expression appears not only in response to EGFR targeting Cetuximab but also in non small cell lung carcinoma (NSCLC) patients treated with Gefitinib the small molecule tyrosine kinase inhibitor. In a NSCLC patient population treated with Gefitinib those with elevated AREG (>93.8 pg/ml) or TGFα (>15.6 pg/ml) levels in serum responded poorly to treatment compared to those patients with low levels (Ishikawa et al., Cancer Res., 2005, 65:9176-9184).

Ovarian tumours have also been described for their dependence on HB-EGF, particularly in the case of aggressive tumours (Tanaka et al., Clin Cancer Res., 2005 Jul. 1; 11(13):4783-92.). Highly sensitive detection methods demonstrated that HB-EGF levels were significantly higher in ovarian cancer patients (28.6 pg/ml) compared to controls (5.4 pg/ml) and levels appeared to increase with later stages of the disease (Kasai et al., Am J Transl Res., 2012; 4(4):415-21).

Resistance to anticancer agents is a major hurdle in the treatment of cancer. Such resistance has resulted in patients becoming cross-resistant to the effects of many different drugs. More particularly, resistance to ErbB therapy is a problem and leads to patient death.

It is thus an object of the invention to provide new cancer treatments that can overcome common mechanisms of resistance such as resistance to ErbB targeted therapies.

A key regulator of the extracellular release of multiple EGFR and ErbB family ligands is the ADAM17 sheddase. ADAM17 has been broadly described for its presence in tumours and its activity either locally or remotely is confirmed by the numerous clinical studies that describe increased ligand release or increased ligand levels. Whereas current therapies aim to target the cellular receptors and the downstream signalling pathways that they activate, targeting ADAM17 will eliminate the source of signalling for the ErbB receptor family. The effect of the silenced signalling will be multi faceted, firstly the direct effect of ligands on receptors and downstream signalling will be silenced, secondly the autocrine loops that result from receptor activation leading to additional ligand expression and shedding will also be silenced, finally a recently emerging resistance mechanism to ErbB targeting therapies that of heterodimer formation will itself be affected as heterodimer signalling is still ligand dependent (Brand et al., Cancer Res, Sep. 15, 2014, 74:5152-5164; Hobor. S et al., 2014; Troiani et al., Clin Cancer Res, Dec. 15, 2013, vol. 19, no. 24, 6751-6765; Wheeler D L et al., 2008). Thus targeting ADAM17 enhances the spectrum of tumours that can be targeted to any of those that have a dependence on ligand activated ErbB signaling, resistance mechansisms to ErbB targeted therapies through the shedding of ErbB ligands, and these effects both locally and systemically dependent on the source of ligand. As previously described the elevated ligand levels observed in ErbB targeting therapies are the by product of the treatment itself, that in turn forces the positive selection of resistant tumours. Targeting ADAM17 removes the stimulatory mechanism from the tumour environment but does not introduce a positive selection pressure for tumour resistance to develop. The absence of ligands could only be considered a negative or neutral selection pressure and thus is much less potent in the directing of resistance development.

ADAM17 (A disintegrin and metalloproteinase domain-containing protein 17) also referred to as Snake venom-like protease, TNF-alpha convertase, TNF-alpha-converting enzyme (TACE) and CD156b is a membrane bound metalloprotease responsible for the extracellular cleavage (ectodomain shedding) of a number of pathologically important substrates. Originally identified as the enzyme responsible for the cleavage of membrane bound pro-TNF-α liberating soluble protein, ADAM17 has since been described in the ectodomain shedding of a large number of membrane bound precursor proteins. Ectodomain shedding by ADAM17 releases from the membrane of cells a large number of soluble cytokines and growth factors such as: Amphiregulin, Heparin binding-EGF like growth factor (HB-EGF), Transforming growth factor alpha (TGF-α), epiregulin, epigen and neuregulins. ADAM17 also mediates the shedding of numerous receptors including; IL-6Rα, IL-1RII, Her4, c-Kit, Notch, Mer, TNF-α RI & II where the physiological result can be signal silencing through receptor shedding, soluble ligand trapping, or receptor transactivation as is described for IL-6Rα and gp130. ADAM17 can actively participate in the remodelling of the extracellular matrix and cell-cell contacts through the shedding of a large number of adhesion molecules and constituents of the extracellular microenvironment such as: L-selectin, ICAM-1, VCAM-1, Nectin-4, CD44 and collagen XVII. Less well understood activities of ADAM17 include the ectodomain shedding of cellular prion protein and amyloid precursor protein.

For the avoidance of doubt, without any specification, the expression ADAM17 refers to the human ADAM17 of sequence SEQ ID No. 29.

Structurally, ADAM17 consists of an 824 amino acid (aa) protein comprising a preproprotein domain (aa 1-214), an extracellular domain (aa 215-671), a transmembrane domain (aa 672-692) and a cytoplasmic domain (aa 693-824).

More particularly, the extracellular domain is comprised of a Metalloprotease (MP) domain of sequence SEQ ID No. 30 (corresponding to aa 215-474 of ADAM17), a Disintegrin (DI) domain of sequence SEQ ID No. 31 (corresponding to aa 475-563 of ADAM17) and a Membrane proximal (MPD) domain of sequence SEQ ID No. 32 (corresponding to aa 564-671 of ADAM17).

It is thus an object of the invention to offer an alternative to existing tumour treatments by providing new tumour treatments of ADAM17 substrate dependant tumours.

The current invention also provides treatments capable of inhibiting the cell surface shedding of ErbB ligands via the targeting of ADAM17.

In a first embodiment, the present application relates to a pharmaceutical composition comprising an effective amount of an ADAM17 antibody, or an antigen-binding fragment thereof, for use in the treatment of ADAM17 substrate dependant tumours, said ADAM17 antibody comprising the following properties:

a) it binds to ADAM17 with a Kd of 3 nM or less;

b) it recognizes an epitope within the membrane proximal domain (MPD) of ADAM17 localized between the residues 564 and 642;

c) it does not bind to ADAM10;

d) it inhibits the cellular shedding of at least one ADAM17 substrate with an IC₅₀ of 200 pM or less;

e) it has an off rate for ADAM17 of K_{off} of 3×10⁻⁴ s⁻¹ or smaller;

f) it inhibits the growth and/or proliferation in vivo of at least one tumour cell expressing ADAM17;

g) it does not bind to the murine ADAM17; and

h) it binds to the cynomolgous ADAM17.

It must be understood here that the invention does not relate to an antibody in a natural form, that is to say it is not in its natural environment but that it has been able to be isolated or obtained by purification from natural sources, or else obtained by genetic recombination, or by chemical synthesis, and that it can then contain unnatural amino acids as will be described herein.

The term “comprising” is meant to be open ended, including the indicated component(s) but not excluding other elements.

As used in the present specification, the expression “ADAM17 antibody” should be interpreted as similar to “anti-ADAM17 antibody” and means an antibody capable of binding to ADAM17. Without any contradictory specification, ADAM17 will be used for the antibody of the present invention including murine, chimeric or humanized ADAM17 antibody.

The terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

In an embodiment of the invention, the pharmaceutical composition comprising the ADAM17 antibody can comprise one or more excipient(s) and/or a pharmaceutical acceptable vehicle(s). The expression “pharmaceutically acceptable vehicle” or “excipient” is intended to indicate a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable vehicles and excipients are well known and will be adapted by the person skilled in the art as a function of the nature and of the mode of administration of the active compound(s) chosen.

An “effective amount” or “therapeutically effective amount” of a compound is that amount of compound which is sufficient to provide a detectable effect (such as a reduction in size or severity of the cancer or tumour) to a cell to which the compound is administered when compared to an otherwise identical cell to which the compound is not administered.

“ADAM17 substrate dependant tumours” are understood to be tumours for which their aberrant growth is inhibited when the serum concentration of one or more ADAM17 substrates is reduced. One skilled in the art can easily establish base line serum levels of ADAM17 substrates using such quantitative techniques as enzyme linked immunosorbent assay (ELISA), Luminex®, electrochemiluminescence or similar approaches.

By “binding”, “binds”, or the like, it is intended that the ADAM17 antibody, or an antigen-binding fragment thereof, forms a complex with an antigen that is relatively stable under physiologic conditions. Methods for determining whether two molecules bind are well known in the art and include, for example, equilibrium dialysis, surface plasmon resonance, and the like. For the avoidance of doubt, it does not mean that the said antibody or antigen-binding fragment could not bind or interfere, at a low level, to another antigen. As a preferred embodiment, the said antibody, or antigen-binding fragment thereof, binds to its antigen with an affinity that is at least two-fold greater than its affinity for binding to a non specific molecule (BSA, casein, etc.). Nevertheless, as another preferred embodiment, the said antibody, or antigen-binding fragment thereof, binds only to the said antigen.

“K_{d}” or “Kd” refers to the dissociation constant of a particular antibody-antigen complex. K_{d}=K_{off}/Kₒₙ with K_{off} consisting in the off rate constant for dissociation of the antibody from an antibody-antigen complex and Kₒₙ consisting in the rate at which the antibody associates with the antigen.

The term “epitope” is a region of an antigen that is bound by an antigen binding protein, including antibodies. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

In a surprising embodiment, the ADAM17 antibody herein described binds to an epitope, also called “ADAM17 epitope”, comprised within the Membrane Proximal Domain (MPD) being itself comprised of amino acid 564-671. More particularly, the said ADAM17 antibody binds to an ADAM17 epitope comprised within the region comprised of amino acids 564-642 of ADAM17 corresponding to a sub-domain of the MPD.

In an embodiment, as demonstrated in the following examples, the ADAM17 epitope of the ADAM17 antibody herein described consists of a portion of the amino acids 564-642 with at least the residue 606 being an aspartic acid (D).

In an embodiment, as demonstrated in the following examples, the ADAM17 epitope of the ADAM17 antibody herein described consists of a portion of the amino acids 564-642 with at least the residue 610 being an arginine (R).

In an embodiment, as demonstrated in the following examples, the ADAM17 epitope of the ADAM17 antibody herein described consists of a portion of the amino acids 564-642 with at least the residue 606 being an aspartic acid (D) and the residue 610 being a arginine (R).

According to a particular aspect, the ADAM17 antibody herein described does not bind to the Metalloprotease (MP) domain, said MP domain being comprised of amino acid 215-474 of ADAM17.

According to a particular aspect, the ADAM17 antibody herein described does not bind to the Disintegrin (DI) domain, said DI domain being comprised of amino acid 475-563 of ADAM17.

According to another aspect, the ADAM17 antibody herein described does not bind, or poorly binds, to the Membrane Proximal Domain (MPD) if the residue 606 is not an aspartic acid (D) and/or the residue 610 is not an arginine (R).

This aspect is surprising as it has never been described, nor suggested, an antagonist, and more particularly an antibody, capable of decreasing or inhibiting the shedding of ADAM17 substrates without interfering with the catalytic domain of ADAM17 which is known to be responsible for the shedding activity. In other words, the ADAM17 antibody herein described is capable of selectively decreasing or inhibiting the enzymatic activity of ADAM17 regarding at least one substrate in the specific context of a pathology, said pathology being cancer. An advantage of the ADAM17 antibody herein described may rely on the fact that it seems to not inhibit the whole catalytic activity of ADAM17 as it does not bind to the catalytic domain, but it may be capable of decreasing or inhibiting the enzymatic activity of ADAM17 for all or part of its substrates.

An antibody binding to the MPD of ADAM17 can be obtained by any of a number of techniques well known to those skilled in the art, including but not limited to, immunisation and hybridoma generation, monoclonal B-cell selection, phage display, ribosomal display, yeast display, expressed immune response sequencing coupled with targeted gene synthesis. Each process can be performed with the ADAM17 protein or a selected sub domain as the target antigen. One skilled in the art could select for MPD binding antibodies from a population of antibodies binding to the ADAM17 extracellular domain, or more particularly the MPD. Subsequent selection and characterisation may also represent the selective step for MPD binding whereby all binders to the ADAM17 extracellular domain are selectively screened for binding to the MPD or selected sub domains of the MPD. Alternatively all selection steps may be performed against the MPD or selected sub domains of the MPD and binding to the native ADAM17 extracellular domain being employed as a subsequent selection and characterisation step.

As used herein, the “shedding inhibition” can be defined as the inhibition of the release of cell surface proteins to the extracellular environment by an enzymatic cleavage of the membrane bound precursor protein, the release being measurable by one skilled in the art by ELISA, Luminex®, electrochemiluminescence or similar approaches, the shedding inhibition being measurable by the same processes.

In an embodiment, the ADAM17 antibody inhibits the cellular shedding of at least one substrate of ADAM17 with an IC₅₀ of 500 pM or less, preferentially 200 pM.

In the context of the invention, the expression “IC₅₀” refers to the concentration of an antibody in a dose response evaluation that is necessary to achieve half the maximal attainable inhibition. Such evaluation of the IC₅₀ can be made by measuring substrate shedding from cells or Fluorescence Resonance Energy Transfer (FRET) peptide cleavage assay with recombinant protein.

For the avoidance of doubt, ADAM17 substrate can be selected from the substrates listed in the following table 1.

TABLE 1

| Gene | Protein |
|---|---|
| ACE2 | Angiotensin-converting enzyme 2 |
| ALCAM | Activated leukocyte cell adhesion molecule |
| AREG | Amphiregulin |
| C4.4A | Ly6/PLAUR domain-containing protein 3 |
| CA9 | Carbonic anhydrase 9 |
| CD163 | Scavenger receptor cysteine-rich type 1 protein M130 |
| CD16a | Low affinity immunoglobulin gamma Fc region |
| | receptor III-A |
| CD16b | Low affinity immunoglobulin gamma Fc region |
| | receptor III-B |
| CD36 | Platelet glycoprotein 4 |
| CD44 | CD44 antigen |
| CD62L | L-selectin |
| CD89 | Immunoglobulin alpha Fc receptor |
| CD91 | Prolow-density lipoprotein receptor-related protein 1 |
| COL17A1 | Collagen alpha-1(XVII) chain |
| CSF1 | Macrophage colony-stimulating factor 1 |
| CSF1R | Macrophage colony-stimulating factor 1 receptor |
| CX3CL1 | Fractalkine |
| DLL1 | Delta-like protein 1 |
| DSG2 | Desmoglein-2 |
| EPCAM | Epithelial cell adhesion molecule |
| EPCR | Endothelial protein C receptor |
| EPGN | Epigen |
| ERBB4 | Receptor tyrosine-protein kinase erbB-4 |
| EREG | Epiregulin |
| F11R | Junctional adhesion molecule A |
| FLT3LG | Fms-related tyrosine kinase 3 ligand |
| GHR | Growth hormone receptor |
| GP1BA | Platelet glycoprotein Ib alpha chain |
| GP5 | Platelet glycoprotein V |
| GP6 | Platelet glycoprotein VI |
| HBEGF | Proheparin-binding EGF-like growth factor |
| ICAM1 | Intercellular adhesion molecule 1 |
| IGF2R | Cation-independent mannose-6-phosphate receptor |
| IL-6Rα | Interleukin-6 receptor subunit alpha |
| JAG1 | Protein jagged-1 |
| KDR | Vascular endothelial growth factor receptor 2 |
| KL | Klotho |
| L1CAM | Neural cell adhesion molecule L1 |
| LAG3 | Lymphocyte activation gene 3 protein |
| OLR1 | Oxidized low-density lipoprotein receptor 1 |
| MET | Hepatocyte growth factor receptor |
| MICA | MHC class I polypeptide-related sequence A |
| MICB | MHC class I polypeptide-related sequence B |
| MUC1 | Mucin-1 |
| NCAM1 | Neural cell adhesion molecule 1 |
| NOTCH1 | Neurogenic locus notch homolog protein 1 |
| NRG1 | Pro-neuregulin-1, membrane-bound isoform |
| NTRK1 | High affinity nerve growth factor receptor |
| PTK7 | Inactive tyrosine-protein kinase 7 |
| PTPRF | Receptor-type tyrosine-protein phosphatase F |
| PTPRZ1 | Receptor-type tyrosine-protein phosphatase zeta |
| PVRL4 | Nectin-4 |
| RANKL | Tumor necrosis factor ligand superfamily member 11 |
| SDC1 | Syndecan-1 |
| SDC4 | Syndecan-4 |
| SEMA4D | Semaphorin-4D |
| TGFA | Protransforming growth factor alpha |
| TMEFF2 | Tomoregulin-2 |
| TNFRSF5 | Tumor necrosis factor receptor superfamily member 5 |
| TNFRSF8 | Tumor necrosis factor receptor superfamily member 8 |
| TNF | Tumor necrosis factor |
| TNFRSF1A | Tumor necrosis factor receptor superfamily member 1A |
| TNFRSF1B | Tumor necrosis factor receptor superfamily member 1B |
| VASN | Vasorin |
| VCAM1 | Vascular cell adhesion protein 1 |

In an embodiment, preferred ADAM 17 substrates are selected from the following groups:

TABLE 2
Adhesion molecules

| | |
|---|---|
| ALCAM | Activated leukocyte cell adhesion molecule |
| CD62L | L-selectin |
| COL17A1 | Collagen alpha-1(XVII) chain |
| EPCAM | Epithelial cell adhesion molecule |
| ICAM1 | Intercellular adhesion molecule 1 |
| L1CAM | Neural cell adhesion molecule L1 |
| MUC1 | Mucin-1 |
| NCAM1 | Neural cell adhesion molecule 1 |
| PVRL4 | Nectin-4 |
| SDC1 | Syndecan-1 |
| SDC4 | Syndecan-4 |
| VCAM1 | Vascular cell adhesion protein 1 |

TABLE 3
Angiogenesis

| | |
|---|---|
| KDR | Vascular endothelial growth factor receptor 2 |

TABLE 4
Chemokine

| | |
|---|---|
| CX3CL1 | Fractalkine |

TABLE 5
Growth Factors

| | |
|---|---|
| AREG | Amphiregulin |
| CSF1 | Macrophage colony-stimulating factor 1 |
| EPGN | Epigen |
| EREG | Epiregulin |
| FLT3LG | Fms-related tyrosine kinase 3 ligand |
| HBEGF | Proheparin-binding EGF-like growth factor |
| NRG1 | Pro-neuregulin-1, membrane-bound isoform |
| NTRK1 | High affinity nerve growth factor receptor |
| TGFA | Protransforming growth factor alpha |

TABLE 6
Growth Factor Receptors

| | |
|---|---|
| CSF1R | Macrophage colony-stimulating factor 1 receptor |
| ERBB4 | Receptor tyrosine-protein kinase erbB-4 |
| IGF2R | Cation-independent mannose-6-phosphate receptor |
| MET | Hepatocyte growth factor receptor |

TABLE 7
Immunomodulatory

| | |
|---|---|
| CD16a | Low affinity immunoglobulin gamma Fc region receptor III-A |
| CD16b | Low affinity immunoglobulin gamma Fc region receptor III-B |
| CD163 | Scavenger receptor cysteine-rich type 1 protein M130 |
| IL-6R□ | Interleukin-6 receptor subunit alpha |
| LAG3 | Lymphocyte activation gene 3 protein |
| MICA | MHC class I polypeptide-related sequence A |
| MICB | MHC class I polypeptide-related sequence B |

TABLE 8
Inflamatory

| | |
|---|---|
| CD36 | Platelet glycoprotein 4 |
| RANKL | Tumor necrosis factor ligand superfamily member 11 |
| TNFRSF5 | Tumor necrosis factor receptor superfamily member 5 |
| TNFRSF8 | Tumor necrosis factor receptor superfamily member 8 |
| TNF | Tumor necrosis factor |
| TNFRSF1A | Tumor necrosis factor receptor superfamily member 1A |
| TNFRSF1B | Tumor necrosis factor receptor superfamily member 1B |
| VASN | Vasorin |

TABLE 9
Metastasis

| | |
|---|---|
| C4.4A | Ly6/PLAUR domain-containing protein 3 |

TABLE 10
Notch Ligands

| | |
|---|---|
| DLL1 | Delta-like protein 1 |
| JAG1 | Protein jagged-1 |

TABLE 11
Tight junctions

| | |
|---|---|
| DSG2 | Desmoglein-2 |
| F11R | Junctional adhesion molecule A |

In another embodiment, preferred ADAM17 substrates are selected from the substrates of the group “Growth Factors” (Table 5).

In still another preferred embodiment, ADAM17 substrates are AREG, HB-EGF (also referred as HBEGF) and TGFα (also referred as TGFA or TGFa).

The invention relates to a pharmaceutical composition for use according to claim 1, wherein the said ADAM17 substrate dependant tumours consist of: (i) tumours characterized by an elevated level of at least one ADAM17 substrate compared to the basal level of said at least one substrate, or (ii) tumours that are resistant or refractory to treatment with an ErbB therapy.

As above mentioned in a first aspect, the said ADAM17 substrate dependant tumours consist of tumours characterized by an elevated level of at least one ADAM17 substrate compared to the basal level of said at least one substrate.

The “basal level” or “base line level” can be established based on population analysis of healthy control or patient samples for levels of ADAM17 substrates. Therapeutic agents that result in reduced serum levels of an ADAM17 substrate or substrates can be determined by quantitative techniques as previously described by comparison of pre and post treatment serum levels of the substrate. The correlation of tumour growth inhibition to reduced serum levels of an ADAM17 substrate or substrates compared to the predetermined base line values serve to determine the existence of an ADAM17 substrate dependant tumour.

“Elevated levels of at least one ADAM17 substrate” should be understood as levels of at least one ADAM17 substrate measured by a quantitative technique such as enzyme linked immunosorbent assay (ELISA), Luminex®, electrochemiluminescence or a similar approach to be at levels at least two fold higher than that established as a base line serum sample of the same ADAM17 substrate from a healthy population. In particular base line HB-EGF levels have been described as 5.4 pg/ml, in a SCCHN population HB-EGF levels were determined at 23 pg/ml, in a recurrent disease population levels were determined at 95 pg/ml. In an ovarian cancer population HB-EGF levels were determined as 28.6 pg/ml. According to an aspect, the ADAM17 antibody binds to ADAM17 with a Kd of about 10 nM or less, preferentially of about 5 nM or less, more preferably of about 2 nM or less, as determined by surface plasmon resonance (SPR). Any other method or technique available to the person skilled in the art may also be used.

As above mentioned in a second aspect, the said ADAM17 substrate dependant tumours consist of tumours that are resistant or refractory to treatment with an ErbB therapy.

As used herein, a tumour that is “refractory” to therapy is one that is initially responsive, becomes unresponsive over time (e.g., within three months (i.e., disease progression may be observed on or within three months of treatment)) or recurs shortly after discontinuation of treatment. In certain embodiments, a “resistant” tumour is also termed a “refractory” tumour.

As used herein, an illness that is “resistant” to therapy is one that is unresponsive to therapy. In one embodiment, the tumour may be resistant at the beginning of treatment or it may become resistant during treatment. In certain embodiments, a “refractory” tumour is also termed a “resistant” tumour.

An “ErbB therapy”, also referred as “ErbB-targeted therapy” or “anti-ErbB therapy”, intends to designate a therapy consisting of administering to a subject a molecule acting as a “ErbB antagonist”, meaning any molecule that binds either to an ErbB receptor or to a ligand and blocks ligand activation of the ErbB receptor. Such antagonists include, but are not limited to, modified ligands, ligand peptides (i.e., ligand fragments), soluble ErbB receptors, and anti-ErbB antibodies.

Another aspect of the invention is a pharmaceutical composition comprising an effective amount of an ADAM17 antibody, or an antigen-binding fragment thereof, for use in the treatment of tumours that are refractory or resistant to treatment with an ErbB therapy, said ADAM17 antibody comprising the following properties:

a) it binds to ADAM17 with a Kd of 3 nM or less;

b) it recognizes an epitope within the membrane proximal domain (MPD) of ADAM17 localized between the residues 564 and 642;

c) it does not bind to ADAM10;

d) it inhibits the cellular shedding of at least one ADAM17 substrate with an IC₅₀ of 200 pM or less;

e) it has an off rate for ADAM17 of K_{off} of 3×10⁻⁴ s⁻¹ or smaller;

f) it inhibits the growth and/or proliferation in vivo of at least one tumour cell expressing ADAM17;

g) it does not bind to the murine ADAM17; and

h) it binds to the cynomolgous ADAM17.

According to an embodiment of the pharmaceutical composition for use according to the invention, the said tumours that are refractory or resistant to treatment with an ErbB therapy consist of: (i) the tumours with elevated levels of ErbB ligands compared to the level before the treatment with an ErbB therapy, or (ii) the tumours with elevated levels of ErbB ligands compared to healthy control.

As illustrative example, without any limitation, the different levels of ErbB ligands before and after treatment can be measured by any method known in the Art such as ELISA, LUMINEX® or electrochemiluminescence.

By the expression “healthy control”, it is intended, for example, to represent a person or population chosen from those lacking an ADAM17 linked pathology.

For more clarity, an ErbB ligand level should be considered as elevated compared to the level before the treatment with an ErbB therapy if the said level is at least two fold higher than the said level before treatment.

In an embodiment of the pharmaceutical composition for use according to the invention, the ErbB therapy comprises administration of an EGFR antibody or an EGFR Kinase inhibitor, a Her2 antibody, or a Her2 kinase inhibitor, a Her3 antibody or a Her3 kinase inhibitor.

Of course, any other ErbB therapy should be considered as encompassed in the present specification. As non limitative example, the ErbB therapy comprises administration of afatinib, erlotinib, gefitinib, lapatinib, icotinib, BIB2992, cetuximab, panitumumab, pertuzumab, zalutumumab, necitumumab, trastuzumab, trastuzumab emtansine and nimotuzumab.

The pharmaceutical composition for use according to the invention is characterized in that the said ADAM17 antibody inhibits the cellular shedding of at least one substrate selected from TNFα, TGFα, AREG, HB-EGF with an IC₅₀ of 500 pM or less.

In an aspect, the ADAM17 antibody is capable of inhibiting the cellular shedding of TNF-α (Tumour necrosis factor alpha), and more preferably with at least an IC₅₀ of 500 pM or less, preferentially 200 pM or less.

In an aspect, the ADAM17 antibody is capable of inhibiting the cellular shedding of TGF-α (Transforming growth factor alpha), and more preferably with at least an IC₅₀ of 500 pM or less, preferentially 200 pM or less.

In an aspect, the ADAM17 antibody is capable of inhibiting the cellular shedding of amphiregulin (AREG), and more preferably with at least an IC₅₀ of 500 pM or less, preferentially 200 pM less.

In an aspect, the ADAM17 antibody is capable of inhibiting the cellular shedding of HB-EGF (Heparin-binding EGF-like growth factor), and more preferably with at least an IC₅₀ of 500 pM or less, preferentially 200 pM or less.

In another embodiment, the ADAM17 antibody is characterized in that it inhibits the cellular shedding of at least one substrate of ADAM17 selected from TNF-α, TGF-α, AREG and HB-EGF with at least an IC₅₀ of 500 pM or less, preferentially 200 pM or less.

The pharmaceutical composition for use according to the invention is characterized in that the said ADAM17 antibody inhibits the cellular shedding of the substrates TNFα, TGFα, AREG and HB-EGF with an IC₅₀ of 500 pM or less.

By the expression “antigen-binding fragment” of an ADAM17 antibody, it is intended to indicate any peptide, polypeptide, or protein retaining the ability to bind to the target (also generally referred to as antigen) of the said ADAM17 antibody, generally the same epitope.

In a preferred embodiment, the said antigen-binding fragment comprises at least one CDR of the ADAM17 antibody from which it is derived. Still in a preferred embodiment, the said antigen-binding fragment comprises 2, 3, 4 or 5 CDRs, more preferably the 6 CDRs of the ADAM17 antibody from which it is derived.

The “antigen-binding fragments” can be selected, without limitation, in the group consisting of Fv, scFv (sc for single chain), Fab, F(ab′)₂, Fab′, scFv-Fc fragments or diabodies, or fusion proteins with disordered peptides such as XTEN (extended recombinant polypeptide) or PAS motifs, or any fragment of which the half-life time would be increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic CDRs of the antibody according to the invention. Preferably, said “antigen-binding fragments” will be constituted or will comprise a partial sequence of the heavy or light variable chain of the antibody from which they are derived, said partial sequence being sufficient to retain the same specificity of binding as the ADAM17 antibody from which it is descended and a sufficient affinity, preferably at least equal to 1/100, in a more preferred manner to at least 1/10, of the affinity of the ADAM17 antibody from which it is descended, with respect to the target.

An embodiment of the pharmaceutical composition for use according to the invention is that said antigen-binding fragment thereof is selected from a Fab fragment, a F(ab′)₂ fragment, a F(ab′) fragment, a scFv fragment, a Fv fragment, a scFv-Fc fragment or a diabody.

An embodiment of the invention of the pharmaceutical composition as above described comprising the ADAM17 antibody, or an antigen-binding fragment thereof, with the following properties:

a) it binds to ADAM17 with a Kd of 3 nM or less;

b) it recognizes an epitope within the membrane proximal domain (MPD) of ADAM17 localized between the residues 564 and 642;

c) it does not bind to ADAM10;

d) it inhibits the shedding of at least one ADAM17 substrate with an IC₅₀ of 200 pM or less;

e) it has an off rate for ADAM17 of K_{off} of 3×10⁻⁴ s⁻¹ or smaller;

f) it inhibits the growth and/or proliferation in vivo of at least one tumour cell expressing ADAM17;

g) it does not bind to the murine ADAM17; and

h) it binds to the cynomologous ADAM17;

said ADAM 17 antibody comprising six CDRs wherein at least one, preferentially at least two, preferentially at least three, preferentially at least four, preferentially at least 5 of the six CDRs are selected from the CDRs of amino acid sequences SEQ ID No. 1 to 6, or any sequence having at least 90% of identity with the SEQ ID No. 1 to 6.

In another embodiment of the invention, the ADAM17 antibody, or any antigen-binding fragment thereof, comprises the six CDRs of amino acid sequences SEQ ID Nos. 1 to 6, or any sequence having at 90% identity with the SEQ ID Nos. 1 to 6.

In an embodiment, the pharmaceutical composition for use according to the invention is characterized in that the said ADAM17 antibody, or an antigen-binding fragment thereof, comprises:

i) a heavy chain domain comprising CDR-H1, CDR-H2 and CDR-H3 of sequence SEQ ID No. 1, 2 and 3, respectively, and

ii) a light chain domain comprising CDR-L1, CDR-L2 and CDR-L3 of sequence SEQ ID No. 4, 5, and 6, respectively.

For the avoidance of doubt, without any contrary indication in the text, the expression CDRs means the hypervariable regions of the heavy and light chains of an antibody as defined by IMGT.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

Three heavy chain CDRs and three light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

In an embodiment, the CDR-H1 comprises the sequence SEQ ID No. 1 wherein the residue referred to as X₁ is selected from polar amino-acids. The polar amino-acid is preferentially selected from asparagine (Asn or N), aspartic acid (Asp or D), glutamine (Gln or Q), serine (Ser or S), glutamic acid (Glu or E), arginine (Arg or R), lysine (Lys or K), histidine (His or H), tryptophan (Trp or W), tyrosine (Tyr or Y) or threonine (Thr or T).

In another preferred embodiment, the residue X₁ is selected from the small size polar amino-acid. The small size polar amino-acid is preferentially selected from asparagine (Asn or N), aspartic acid (Asp or D), serine (Ser or S) or threonine (Thr or T).

In another embodiment, the residue X₁ is asparagine (Asn or N).

In another embodiment, the residue X₁ is aspartic acid (Asp or D).

In an embodiment, the pharmaceutical composition for use according to the invention is characterized in that the CDR-H1 is of sequence SEQ ID No. 7 or 8.

In the sense of the present invention, the “percentage identity” or “% identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid, nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/b12.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

For the amino acid sequence exhibiting at least 90% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antibodies likely to be generated.

As a non-limiting example, table 12 below summarizes the possible substitutions likely to be carried out without resulting in a significant modification of the biological activity of the corresponding modified antibody; inverse substitutions are naturally possible under the same conditions.

TABLE 12

| Original residue | Substitution(s) |
|---|---|
| Ala (A) | Val, Gly, Pro, Ser, Thr |
| Arg (R) | Lys, His, Gln |
| Asn (N) | Gln, Asp, His, Lys, Ser, Thr |
| Asp (D) | Glu, Asn |
| Cys (C) | Ser |
| Gln (Q) | Asn, Arg, Glu, His, Lys, Met |
| Glu (G) | Asp, Gln, Lys |
| Gly (G) | Ala, Pro |
| His (H) | Arg, Asn, Gln, Tyr |
| Ile (I) | Leu, Val, Met |
| Leu (L) | Ile, Val, Met, Phe |
| Lys (K) | Arg, Gln, Glu, Asn |
| Met (M) | Leu, Ile,, Gln, Val |
| Phe (F) | Tyr, Met, Leu, Trp |
| Pro (P) | Ala |
| Ser (S) | Thr, Cys, Ala, Asn |
| Thr (T) | Ser, Ala, Asn |
| Trp (W) | Tyr, Phe |
| Tyr (Y) | Phe, Trp, His |
| Val (V) | Leu, Ala, Ile, Met |

The ADAM17 antibody, or any antigen-binding fragment thereof, can also be described as comprising: i) a heavy chain comprising CDR-H1, CDR-H2 and CDR-H3 comprising respectively amino acid sequences SEQ ID Nos. 7, 2 and 3, or sequences with at least 80%, preferably 85%, 90%, 95% and 98% identity after optimal alignment with sequences SEQ ID Nos. 7, 2 and 3; and ii) a light chain comprising CDR-L1, CDR-L2 and CDR-L3 comprising respectively amino acid sequences SEQ ID Nos. 4, 5 and 6, or sequences with at least 80%, preferably 85%, 90%, 95% and 98% identity after optimal alignment with sequences SEQ ID Nos. 4, 5 and 6.

The ADAM17 antibody, or any antigen-binding fragment thereof, can also be described as comprising: i) a heavy chain comprising CDR-H1, CDR-H2 and CDR-H3 comprising respectively amino acid sequences SEQ ID Nos. 8, 2 and 3, or sequences with at least 80%, preferably 85%, 90%, 95% and 98% identity after optimal alignment with sequences SEQ ID Nos. 8, 2 and 3; and ii) a light chain comprising CDR-L1, CDR-L2 and CDR-L3 comprising respectively amino acid sequences SEQ ID Nos. 4, 5 and 6, or sequences with at least 80%, preferably 85%, 90%, 95% and 98% identity after optimal alignment with sequences SEQ ID Nos. 4, 5 and 6.

According to still another embodiment, the ADAM17 antibody, or an antigen-binding fragment thereof, comprises a heavy chain variable domain of sequence comprising the amino acid sequence SEQ ID No. 9 or a sequence with at least 80%, preferably 85%, 90%, 95% and 98% identity after optimal alignment with sequence SEQ ID No. 9.

According to still another embodiment, the ADAM17 antibody, or an antigen-binding fragment thereof, comprises a heavy chain variable domain of sequence comprising the amino acid sequence SEQ ID No. 11 or a sequence with at least 80%, preferably 85%, 90%, 95% and 98% identity after optimal alignment with sequence SEQ ID No. 11.

According to still another embodiment, the ADAM17 antibody, or an antigen-binding fragment thereof, comprises a heavy chain variable domain of sequence comprising the amino acid sequence SEQ ID No. 12 or a sequence with at least 80%, preferably 85%, 90%, 95% and 98% identity after optimal alignment with sequence SEQ ID No. 12.

According to still another embodiment, the ADAM17 antibody, or an antigen-binding fragment thereof, comprises a light chain variable domain of sequence comprising the amino acid sequence SEQ ID No. 10 or a sequence with at least 80%, preferably 85%, 90%, 95% and 98% identity after optimal alignment with sequence SEQ ID No. 10.

According to still another embodiment, the ADAM17 antibody, or an antigen-binding fragment thereof, comprises
i) a heavy chain variable domain of sequence comprising the amino acid sequence selected from SEQ ID No. 9, 11 or 12 or a sequence with at least 90% identity after optimal alignment with sequence SEQ ID No. 9, 11 or 12, and
ii) a light chain variable domain of sequence comprising the amino acid sequence SEQ ID No. 10 or a sequence with at least 90% identity after optimal alignment with sequence SEQ ID No. 10.

In another embodiment, the ADAM17 antibody consists of a chimeric antibody. In this case, it can also be referred as c1022C3.

In an embodiment, the ADAM17 antibody consists of a humanized antibody. In this case, it can also be referred as hz1022C3.

In an embodiment, the ADAM17 antibody consists of a human antibody. In this case, it can also be referred as h1022C3.

For the avoidance of doubt, 1022C3, without any prefix, should be considered as encompassing m1022C3, c1022C3, h1022C3 and hz1022C3. In the same sense, 1022C3, m1022C3, c1022C3, h1022C3 and hz1022C3 are all encompassed by the expression ADAM17 antibody.

For more clarity, table 13 below summarizes the various amino acid sequences corresponding to the ADAM17 antibody.

TABLE 13

| | | | | SEQ |
|---|---|---|---|---|
| | CDR | | | ID |
| Antibody | numbering | Heavy chain | Light chain | NO. |
| 1022C3 | IMGT | CDR-H1 (X₁) | | 1 |
| | | CDR-H1 (N) | | 7 |
| | | CDR-H1 (D) | | 8 |
| | | CDR-H2 | | 2 |
| | | CDR-H3 | | 3 |
| | | | CDR-L1 | 4 |
| | | | CDR-L2 | 5 |
| | | | CDR-L3 | 6 |
| | | variable domain (X₁) | | 9 |
| | | variable domain (N) | | 11 |
| | | variable domain (D) | | 12 |
| | | | variable domain | 10 |
| | | Chimeric (c) IgG1* | | 33 |
| | | Chimeric (c) IgG1 | | 34 |
| | | AlaAla* | | |
| | | | Chimeric (c) | 35 |
| | | | IgG1 | |
| | | Humanized (hz) Cons | | 39 |
| | | Hz variable domain | | 40 |
| | | Hz full IgG1* | | 41 |
| | | Hz full IgG2* | | 42 |
| | | Hz full IgG3* | | 43 |
| | | Hz full IgG4*/** | | 44 |
| | | | Humanized (hz) | 45 |
| | | | Cons | |
| | | | Hz variable | 46 |
| | | | domain | |
| | | | Hz full | 47 |

*sequences without the C-terminal lysine (K) residue
**stabilized hinge region serine (S)/proline (P) conversion.

In a particular aspect, the ADAM17 antibody, or an antigen-binding fragment thereof, consists of a chimeric antibody.

A chimeric antibody is one containing a natural variable region (light chain and heavy chain) derived from an antibody of a given species in combination with constant regions of the light chain and the heavy chain of an antibody of a species heterologous to said given species.

The antibodies, or chimeric fragments of same, can be prepared by using the techniques of recombinant genetics. For example, the chimeric antibody could be produced by cloning recombinant DNA containing a promoter and a sequence coding for the variable region of a nonhuman monoclonal antibody of the invention, notably murine, and a sequence coding for the human antibody constant region. A chimeric antibody according to the invention coded by one such recombinant gene could be, for example, a mouse-human chimera, the specificity of this antibody being determined by the variable region derived from the murine DNA and its isotype determined by the constant region derived from human DNA. Refer to Verhoeyn et al. (BioEssays, 8:74, 1988) for methods for preparing chimeric antibodies.

A specific aspect of the invention relates to an ADAM17 antibody, or an antigen-binding fragment thereof, said ADAM17 antibody consisting of a chimeric antibody selected from:
i) a chimeric ADAM17 antibody comprising a) a heavy chain variable region with CDR-H1, CDR-H2 and CDR-H3 comprising respectively amino acid sequences SEQ ID Nos. 1, 2 and 3, or sequences with at least 90% identity with SEQ ID Nos. 1, 2 and 3; and b) a light chain variable region with CDR-L1, CDR-L2 and CDR-L3 comprising respectively amino acid sequences SEQ ID Nos. 4, 5 and 6, or sequences with at least 90% identity with SEQ ID Nos. 4, 5 and 6; and c) light-chain and heavy-chain constant regions derived from an antibody of a species heterologous with the mouse;
ii) a chimeric ADAM17 antibody comprising a heavy chain variable domain of sequence SEQ ID No. 9, 11 or 12 or a sequence with at least 90% identity with SEQ ID No. 9, 11 or 12 and/or a light chain variable domain of sequence SEQ ID No. 10 or a sequence with at least 90% identity with SEQ ID No. 10;
iii) a chimeric ADAM17 antibody comprising a heavy chain domain of sequence SEQ ID No. 33 or a sequence with at least 90% identity with SEQ ID No. 33 and/or a light chain domain of sequence SEQ ID No. 35 or a sequence with at least 90% identity with SEQ ID No. 35; and
iv) a chimeric ADAM17 antibody comprising a heavy chain domain of sequence SEQ ID No. 34 or a sequence with at least 90% identity with SEQ ID No. 34 and/or a light chain domain of sequence SEQ ID No. 35 or a sequence with at least 90% identity with SEQ ID No. 35.

The pharmaceutical composition for use according to the invention is characterized in that i) the said chimeric ADAM17 antibody, or an antigen-binding fragment thereof, comprises a heavy chain variable domain of sequence SEQ ID No. 9, 11 or 12 and/or a light chain variable domain of sequence SEQ ID No. 10; or ii) the said chimeric ADAM17 antibody, or an antigen binding fragment thereof, comprises a heavy chain domain of sequence SEQ ID No. 33 or 34 and/or a light chain domain of sequence SEQ ID No. 35.

In an embodiment, the said species heterologous with the mouse is human (also possibly referred to as man).

In a particular aspect, the ADAM17 antibody, or an antigen-binding fragment thereof, consists of a humanized antibody.

“Humanized antibody” means an antibody that contains CDR regions derived from an antibody of nonhuman origin, the other parts of the antibody molecule being derived from one (or several) human antibodies. In addition, some of the skeleton segment residues (called FR) can be modified to preserve binding affinity (Jones et al., Nature, 321:522-525, 1986; Verhoeyen et al., Science, 239:1534-1536, 1988; Riechmann et al., Nature, 332:323-327, 1988).

The humanized antibodies of the invention or fragments of same can be prepared by techniques known to a person skilled in the art (such as, for example, those described in the documents Singer et al., J. Immun., 150:2844-2857, 1992; Mountain et al., Biotechnol. Genet. Eng. Rev., 10:1-142, 1992; and Bebbington et al., Bio/Technology, 10:169-175, 1992). Such humanized antibodies are preferred for their use in methods involving in vitro diagnoses or preventive and/or therapeutic treatment in vivo. Other humanization techniques, also known to a person skilled in the art, such as, for example, the “CDR grafting” technique described by PDL in patents EP 0 451 261, EP 0 682 040, EP 0 939 127, EP 0 566 647 or U.S. Pat. Nos. 5,530,101, 6,180,370, 5,585,089 and U.S. Pat. No. 5,693,761. U.S. Pat. Nos. 5,639,641 or 6,054,297, 5,886,152 and 5,877,293 can also be cited.

A specific aspect of the invention relates to an ADAM17 antibody, or an antigen-binding fragment thereof, said ADAM17 antibody consisting of a humanized antibody selected from:
i) a humanized ADAM17 antibody comprising a) a heavy chain variable region with CDR-H1, CDR-H2 and CDR-H3 comprising respectively amino acid sequences SEQ ID Nos. 1, 2 and 3, or sequences with at least 90% identity with SEQ ID Nos. 1, 2 and 3; and b) a light chain variable region with CDR-L1, CDR-L2 and CDR-L3 comprising respectively amino acid sequences SEQ ID Nos. 4, 5 and 6, or sequences with at least 90% identity with SEQ ID Nos. 4, 5 and 6;
ii) a humanized ADAM17 antibody comprising a heavy chain variable domain of sequence 39 or 40 or a sequence with at least 90% identity with SEQ ID No. 39 or 40 and/or a light chain variable domain of sequence SEQ ID No. 45 or 46 or a sequence with at least 90% identity with SEQ ID No. 45 or 46;
iii) a humanized ADAM17 antibody comprising a heavy chain domain of sequence SEQ ID No. 41 or a sequence with at least 90% identity with SEQ ID No. 41 and/or a light chain domain of sequence SEQ ID No. 47 or a sequence with at least 90% identity with SEQ ID No. 47;
iv) a humanized ADAM17 antibody comprising a heavy chain domain of sequence SEQ ID No. 42 or a sequence with at least 90% identity with SEQ ID No. 42 and/or a light chain domain of sequence SEQ ID No. 47 or a sequence with at least 90% identity with SEQ ID No. 47;
v) a humanized ADAM17 antibody comprising a heavy chain domain of sequence SEQ ID No. 43 or a sequence with at least 90% identity with SEQ ID No. 43 and/or a light chain domain of sequence SEQ ID No. 47 or a sequence with at least 90% identity with SEQ ID No. 47; and
vi) a humanized ADAM17 antibody comprising a heavy chain domain of sequence SEQ ID No. 44 or a sequence with at least 90% identity with SEQ ID No. 44 and/or a light chain domain of sequence SEQ ID No. 47 or a sequence with at least 90% identity with SEQ ID No. 47.

The pharmaceutical composition for use according to the invention is characterized in that i) the said humanized ADAM17 antibody, or an antigen-binding fragment thereof, comprises a heavy chain variable domain of sequence SEQ ID No. 39 or 40 and/or a light chain variable domain of sequence SEQ ID No. 45 or 46; or the said humanized ADAM17 antibody, or an antigen binding fragment thereof, comprises a heavy chain domain of sequence SEQ ID No. 41, 42, 43 or 44 and/or a light chain domain of sequence SEQ ID No. 37.

In an embodiment, the pharmaceutical composition for use according to the invention is characterized in that the said ADAM17 antibody, or an antigen-binding fragment thereof, is selected from:

(i) a chimeric antibody comprising a heavy chain variable domain of sequence SEQ ID No. 9, 11 or 12 and/or a light chain variable domain of sequence SEQ ID No. 10;

(ii) a chimeric antibody comprising a heavy chain domain of sequence SEQ ID No. 33 or 34 and/or a light chain domain of sequence SEQ ID No. 35;

(iii) a humanized antibody comprising a heavy chain variable domain of sequence SEQ ID No. 39 or 40 and/or a light chain variable domain of sequence SEQ ID No. 45 or 46; or

(iv) a humanized antibody comprising a heavy chain domain of sequence SEQ ID No. 41, 42, 43 or 44 and/or a light chain domain of sequence SEQ ID No. 37.

The invention also relates to an ADAM17 antibody named 1022C3, or an antigen-binding fragment thereof, comprising:

i) a heavy chain domain comprising CDR-H1, CDR-H2 and CDR-H3 of sequence SEQ ID No. 1, 2 and 3, respectively, and

ii) a light chain domain comprising CDR-L1, CDR-L2 and CDR-L3 of sequence SEQ ID No. 4, 5, and 6, respectively, for use in the treatment of ADAM17 substrate dependant tumours.

It is also encompass the ADAM17 antibody for use as above described, wherein the antibody consists of the c1022C3 or the hz1022C3.

The ADAM17 antibody, or an antigen-binding fragment thereof, characterized in that as above stipulated may consist of the monoclonal antibody 1022C3 obtained from the hybridoma 1-4686 deposited at the CNCM, Institut Pasteur, 25 Rue du Docteur Roux, 75725 Paris Cedex 15, France, on the 18 Oct. 2012. Said hybridoma was obtained by the fusion of Balb/C immunized mice splenocytes and cells of the myeloma Sp 2/O-Ag 14 lines.

In other words, the invention also relates to a murine, chimeric, humanized or human ADAM17 antibody, or an antigen-binding fragment thereof, comprising:
i) the amino acid sequence of the heavy chain domain of the antibody expressed by the hybridoma cell line 1-4686 deposited at the CNCM; and
ii) the amino acid sequence of the light chain domain of the antibody expressed by the hybridoma cell line 1-4686 deposited at the CNCM.

An object of the scope of the present invention is an antibody for use in the treatment of ADAM17 substrate dependant tumours, wherein it consists of an affinity matured mutant of the ADAM17 antibody described.

In a preferred embodiment, the said affinity matured mutant consists of a mutant having higher affinity as compared to the said initial ADAM17 antibody.

Any method known by the person skilled in the art should be used for affinity maturation. As non limitative example, it can be mentioned targeted or random mutagenesis of the variable domains, targeted or random mutagenesis of the CDR(s), chain shuffling with antibody libraries or novel heavy or light chains, cellular amelioration or other similarly appropriate methods followed by selection and screening for clones of higher affinity.

It is also an object of the invention to claim a method of inhibiting the growth of tumour cells that are refractory or resistant to ErbB therapy in a subject, wherein the said method comprises contacting said tumour cells with an effective amount of an ADAM17 antibody, or an antigen-binding fragment thereof, said ADAM17 antibody comprising the following properties:

a) it binds to ADAM17 with a Kd of 3 nM or less;

b) it recognizes an epitope within the membrane proximal domain (MPD) of ADAM17 localized between the residues 564 and 642;

c) it does not bind to ADAM10;

d) it inhibits the cellular shedding of at least one ADAM17 substrate with an IC₅₀ of 200 pM or less;

e) it has an off rate for ADAM17 of K_{off} of 3×10⁻⁴ s⁻¹ or smaller;

f) it inhibits the growth and/or proliferation in vivo of at least one tumour cell expressing ADAM17;

g) it does not bind to the murine ADAM17; and

h) it binds to the cynomolgous ADAM17.

The term “subject” as used herein refers to any mammal, including humans and animals, such as cows, horses, dogs and cats. Thus, the invention may be used in human patients as well as in veterinarian subjects and patients. In one embodiment of the invention, the subject is a human.

In a particular embodiment, the method according to the invention is characterized in that the said ADAM17 antibody inhibits the cellular shedding of at least one substrate selected from TNFα, TGFα, AREG, HB-EGF with an IC₅₀ of 500 pM or less.

In another particular embodiment, the method according to the invention is characterized in that the said ADAM17 antibody inhibits the cellular shedding of the substrates TNFα, TGFα, AREG and HB-EGF with an IC₅₀ of 500 pM or less.

As it has been previously mentioned, an aspect of the method according to the invention is that the said tumours that are refractory or resistant to treatment with an ErbB therapy consist of (a) the tumours characterized by elevated levels of ErbB ligands compared to the level before the treatment with an ErbB therapy, or (b) the tumours characterized by elevated levels of ErbB ligands compared to healthy control.

In an embodiment, non limitative, the method according to the invention is characterized in that the ErbB therapy comprises administration of an EGFR antibody or an EGFR Kinase inhibitor, a Her2 antibody, or a Her2 kinase inhibitor, a Her3 antibody, a Her3 kinase inhibitor. As a preferred example, the ErbB therapy comprises administration of afatinib, erlotinib, gefitinib, lapatinib, icotinib, BIB2992, cetuximab, panitumumab, pertuzumab, zalutumumab, necitumumab, trastuzumab, trastuzumab emtansine and nimotuzumab.

It is also described in the present specification a method of inhibiting the growth of tumour cells that are refractory or resistant to ErbB therapy in a subject, characterized in that the said method comprises contacting said tumour cells with an effective amount of an ADAM17 antibody, or an antigen-binding fragment thereof, which comprises:

i) a heavy chain domain comprising CDR-H1, CDR-H2 and CDR-H3 of sequence SEQ ID No. 1, 2 and 3, respectively, and

ii) a light chain domain comprising CDR-L1, CDR-L2 and CDR-L3 of sequence SEQ ID No. 4, 5, and 6, respectively.

According to an embodiment, the CDR-H1 of the ADAM17 antibody is of sequence SEQ ID No. 7 or 8.

Another embodiment of the invention is a method wherein the said ADAM17 antibody, or an antigen-binding fragment thereof, consists of:

(i) a chimeric antibody comprising a heavy chain variable domain of sequence SEQ ID No. 9, 11 or 12 and/or a light chain variable domain of sequence SEQ ID No. 10;

(ii) a chimeric antibody comprising a heavy chain domain of sequence SEQ ID No. 33 or 34 and/or a light chain domain of sequence SEQ ID No. 35;

(iii) a humanized antibody comprising a heavy chain variable domain of sequence SEQ ID No. 39 or 40 and/or a light chain variable domain of sequence SEQ ID No. 45 or 46; or

(iv) a humanized antibody comprising a heavy chain domain of sequence SEQ ID No. 41, 42, 43 or 44 and/or a light chain domain of sequence SEQ ID No. 37.

Other characteristics and advantages of the invention appear further in the description with the examples and figures whose legends are presented below.

### LEGEND OF THE FIGURES

FIG. 1: Effect of 1022C3 on TGFα-Nluc release of A431-TGFα-Nluc cells.

FIG. 2: Effect of 1022C3 on AREG-Nluc release of A431-AREG-Nluc cells.

FIG. 3: Effect of 1022C3 on TNFα-Nluc release of A431-TNFα-Nluc cells.

FIG. 4: Effect of 1022C3 on HB-EGF-Nluc release of A431-HB-EGF-Nluc cells.

FIG. 5: FRET peptide cleavage assay for 1022C3 variants.

FIG. 6: Binding of 1022C3 glycosylated and enzymatically deglycosylated to tumour cell line NCI-H1299.

FIG. 7: Binding ELISA of 1022C3 and Ab936 (polyclonal anti <human ADAM10) to recombinant human (rh) ADAM17 and rhADAM10.

FIGS. 8a and 8b: Comparison of the murine 1022C3 (m1022C3) with its humanized form (hz1022C3) on the CaOV3 xenograft model when used at 1.25 mg/kg (FIG. 8a) and when used at 5 mg/kg (FIG. 8b).

FIG. 9: A431 WT cells treated with 1022C3 or m225.

FIG. 10: A431-AREG cells treated with 1022C3 or m225.

FIG. 11: A431-AREG cells treated with ADAM17 antibodies 1022C3 or 1040H5

FIG. 12: A431-HB-EGF cells treated with 1022C3 or m225.

FIG. 13: A431-HBEGF cells treated with ADAM17 antibodies 1022C3 or 1040H5.

FIG. 14: A431-AREG (low m225 responding model) large tumour volume cells treated with 1022C3 from day 20.

FIG. 15: A431-HBEGF (m225 resistant model) large tumour volume cells treated with 1022C3 from day 20.

FIG. 16: A431-AREG (low m225 responding model) cells treated with 1022C3 as a second line therapy from day 20.

FIG. 17: A431-HBEGF (m225 resistant model) cells treated with 1022C3 as a second line therapy from day 20.

### EXAMPLE 1: GENERATION OF THE ANTIBODY

To generate murine monoclonal antibodies (mAbs) against human ADAM17, 5 BALB/c mice were immunized 3-times subcutaneously with 15-20 μg of the human ADAM17 recombinant protein (R and D Systems, ref: 930-ADB, rhADAM17). The first immunization was performed in the presence of Complete Freund's Adjuvant (Sigma, St Louis, Md., USA). Incomplete Freund's adjuvant (Sigma) was added for following immunizations.

Three days prior to the fusion, 2 immunized mice (selected based on sera titration) were boosted with 15-20 μg of rhADAM17 protein with incomplete Freund's adjuvant. Lymphocytes were prepared by mincing of the proximal lymph nodes, they were then fused to SP2/0-Ag14 myeloma cells in a 1:4 ratio (lymphocyte:myeloma) (ATCC, Rockville, Md., USA). The fusion protocol is that described by Kohler and Milstein (1975), finally, 50 96 well plates were seeded. Fused cells were then subjected to metabolic HAT selection. Approximately 10 days after the fusion, colonies of hybrid cells were screened. For the primary screen, supernatants of hybridomas were evaluated for the secretion of mAbs raised against human ADAM17 using an ELISA.

Briefly, 96-well ELISA plates (Costar 3690, Corning, N.Y., USA) were coated with 50 μl/well of the recombinant human ADAM17 protein (R and D Systems, ref: 930 ADB) at 0.7 μg/ml in PBS overnight at 4° C. The plates were then blocked with PBS containing 0.5% gelatin (#22151, Serva Electrophoresis GmbH, Heidelberg, Germany) for 2 h at 37° C. Once the saturation buffer discarded by flicking plates, 50 μl of sample (hybridoma supernatant or purified antibody) was added to the ELISA plates and incubated for 1 h at 37° C. After three washes, 50 μl horseradish peroxidase-conjugated polyclonal goat anti-mouse IgG (#115-035-164, Jackson Immuno-Research Laboratories, Inc., West Grove, Pa., USA) was added at a 1/5000 dilution in PBS containing 0.1% gelatin and 0.05% Tween 20 (w:w) for 1 h at 37° C. ELISA plates were washed 3-times and TMB (#UP664782, Uptima, Interchim, France) substrate was added. After a 10 min incubation time at room temperature, the reaction was stopped using 1 M sulfuric acid and the optical density at 450 nm was measured.

As a second screening step, selected hybridoma supernatants were evaluated by FACS analysis for mAbs able to bind the cellular form of ADAM17 expressed on the surface of A172 human tumour cells. For the selection by flow cytometry, 2×10⁵ cells were plated in each well of a 96 well-plate in PBS containing 1% BSA and 0.01% sodium azide (FACS buffer) at 4° C. After a 2 min centrifugation at 2000 rpm, the buffer was removed and hybridoma supernatants to be tested were added. After 20 min of incubation at 4° C., cells were washed twice and an Alexa 488-conjugated goat anti-mouse antibody diluted 1/500 in FACS buffer (#A11017, Molecular Probes Inc., Eugene, USA) was added and incubated for 20 min at 4° C. After a final wash with FACS buffer, cells were analyzed by FACS (Facscalibur, Becton-Dickinson) after addition of propidium iodide to each tube at a final concentration of 40 μg/ml. Wells containing cells alone and cells incubated with the secondary Alexa 488-conjugated antibody were included as negative controls. Isotype controls were used in each experiment (Sigma, ref M90351MG). At least 5000 cells were assessed to calculate the mean value of fluorescence intensity (MFI).

As soon as possible, selected hybridomas were cloned by limiting dilution. One 96-well plate was prepared for each code. A volume of 100 μl of a cell suspension adjusted to 8 cells/ml in cloning specific culture medium was loaded in each well. At Day 7, the wells were microscopically examined to ensure cloning and plating efficiency before refeeding the plates with 100 μl of cloning specific culture medium. At days 9-10, the hybridoma supernatants were subsequently screened for their reactivity against the rhADAM17 protein. Cloned mAbs were then isotyped using an Isotyping kit (cat #5300.05, Southern Biotech, Birmingham, Ala., USA). One clone obtained from each hybridoma was selected and expanded to confirm their binding specificity against rhADAM17 and human tumour cells (A172).

### EXAMPLE 2: ADAM 17 SHEDDING OF RECOMBINANT SUBSTRATES FROM TUMOUR CELL LINE A431

Stably transfected A431 cell lines, expressing at their plasma membrane pro-TGFα, pro-HB-EGF, pro-amphiregulin or a mutated pro-TNFα each fused to NanoLuc® Luciiferase (Promega), were generated. ADAM17 activity at the plasma membrane of these cells resulted in the release in the culture medium of the mature substrates fused to NanoLuc® Luciferase. Time dependant measurements of NanoLuc® Luciferase (NLuc) activity in culture medium samples reflected ADAM17 activity. A431 substrate-Nluc cells were seeded at 30 000 cells/well in a 96 wells culture plate. Two days later, culture medium was removed and replaced by 200 μl of fresh culture medium in which were diluted different concentrations of the anti-ADAM17 mAb (1022C3) or the irrelevant mAb (9G4). After 24 h of culture (37° C., CO₂ 5%) 5 μl of culture medium from all experimental wells was collected and distributed in wells of white half-area 96 well plates. After addition of 15 μl of (PBS diluted) Nano-Glo™ luciferase substrate (furimazine), total luminescence for each experimental was read during 0.1 s on a Berthold Mithras LB940 multimode microplate reader.

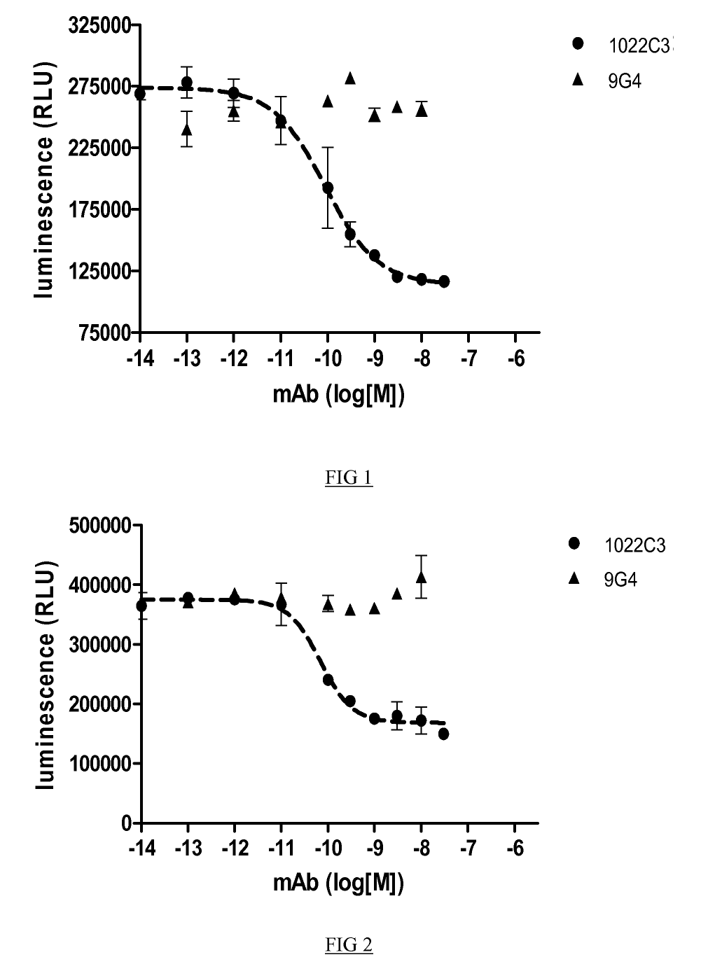

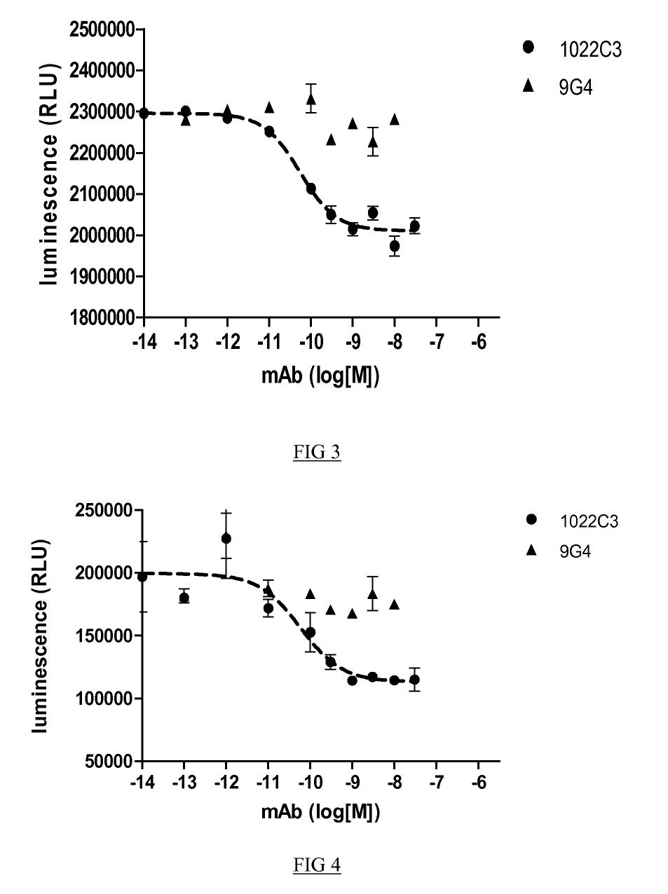

The 1022C3 induced a dose-dependant decrease of i) TGFα-Nluc release in culture medium (FIG. 1), ii) AREG-Nluc release in culture medium (FIG. 2), iii) TNFα-Nluc release in culture medium (FIG. 3) and iv) HB-EGF-Nluc release in culture medium (FIG. 4).

### EXAMPLE 3: mAb 1022C3 BINDING TO ADAM17

The binding profile of 1022C3 to human, murine and chimeric ADAM17 was determined by western blot and surface Plasmon resonance. A number of ADAM17 sub domains and human/murine chimeric proteins were expressed as human Fc fusion proteins from HEK293 cells. Protein A purified proteins were tested for binding following SDS-PAGE separation followed by western blot analysis with 1022C3 and by surface plasmon resonance. The proteins produced and tested for binding are detailed in table 4. Amino acid positions are cited with reference to human ADAM17: accession number P78536 and murine ADAM17: accession number AAI38421. Expressed ADAM17 domains of human origin are written in uppercase letters, domains or murine origin are in lower case letters. Domain names are abbreviated as follows: P, pro-domain; C, catalytic domain; D, disintegrin domain; MPD, membrane proximal domain. Fragmented domains are indicated by their position in the protein structure amino-terminally (Nter) or carboxy-terminally (Cter).

Western Blot Binding Assay:

Equal amounts of purified proteins were resolved by 4-15% SDS-polyacrylamide gel under non reducing conditions and transferred to nitrocellulose membrane. Blocking was performed by incubating the membrane with 1% non fat milk in Tris-buffered saline (TBS) containing 0.05% Tween 20 (TBS-T). The membrane was then incubated with 1 μg/ml 1022C3 antibody in TBS-T for 1 h at room temperature under continuous agitation and then with horseradish peroxidase-conjugated anti-mouse IgG at a dilution of 1:3000 in TBS-T for 1 h at room temperature under continuous agitation. Immunoreactive proteins were visualized by enhanced chemiluminescent detection system kit according to the manufacturer's instructions.

BIAcore Binding Assay:

The experiment was performed on a Biacore X100 device. The 1022C3 is used as the ligand and the ADAM17 fragments and chimeric constructs are used as the analyte. The experiment is run at 10 μl/min at 25° C. on a rabbit anti-mouse polyclonal antibody (Mouse antibody capture kit, BR-1008-38, GE Healthcare) covalently linked to the matrix of both flowcells of a CM5 sensorchip (BR-1000-12) using the amine coupling kit (BR-1000-50, GE Healthcare), using the HBS-EP+ buffer (BR-1008-26, GE Healthcare) as the running buffer. This buffer is also used for the dilutions of the ligand and the analytes.

A solution of the 1022C3 at the concentration of 15 μg/ml is injected on the second flowcell (working surface) during 1 minute. At each cycle, one of the ADAM17 chimeric constructions (with a human Fc domain at the c-terminal positions) is injected at the concentration of 250 nM during 3 minutes on both flow cells: the reference without any 1022C3 (FC1) and the working cell with around 700 RU of m1022C3 (FC2). The registered signal corresponds to the difference between FC2 and FC1 responses.

The positive response is between 90 and 140 RU. The negative responses are all bellow 10 RU. At the end of each cycle the 1022C3 is removed by a injection of a 10 mM Glycine, HCl pH 1.7 buffer (from the Mouse antibody captured kit) during 3 minutes.

TABLE 14

| Amino acid position | Domain | WB | BIAcore |
|---|---|---|---|
| H1-671 | P-C-D-MPD | + | + |
| M1-671 | p-c-d-mpd | − | − |
| H1-474 | P-C | − | − |
| H223-563 | C-D | − | − |
| H475-563 | D | − | − |
| H223-602 | C-D MPD (Nter) | − | − |
| H475-602 | D-MPD (Nter) | − | − |
| H457-671 | D-MPD | + | + |
| H603-671 | MPD (Cter) | − | − |
| M1-602H603-671 | p-c-d-mpd (Nter)-MPD (Cter) | − | − |
| M1-563H564-671 | p-c-d-MPD | + | + |
| M1-474H475-671 | p-c-D-MPD | + | + |

### EXAMPLE 4: DEFINITION OF THE DISSOCIATION CONSTANT OF THE BINDING OF THE EXTRACELLULAR DOMAIN OF ADAM-17 ON MONOCLONAL ANTIBODY 1022C3 WITH SURFACE PLASMON RESONANCE EXPERIMENTS

The antibody (ligand) was bound to the second flowcell of a Biacore CM5 sensor chip (GE Healthcare) activated on both flowcells with a Rabbit anti-Mouse (RAM) IgG (H+L) covalently linked to the carboxymethyldextran matrix. Soluble ADAM17 (analyte) at concentrations ranging from 400 to 12.5 nM obtained by a two fold dilution scheme (assuming a molecular weight of 52 kDa) was injected onto the surface at a flow rate of 30 μl/min in a 120 s pulse (association) plus an extra 180 s delay for the dissociation phase measurement. The RAM surface was regenerated using NaOH 30 mM, NaCl 150 mM and 10 mM Glycine, HCl pH 1.5 buffer solutions. Curves obtained at each concentration were double referenced by first subtracting the signal from the reference FC1 surface (RAM without any mouse anti-TACE mAb) followed by subtraction of the signal obtained from a running buffer injection (Biacore HBS-EP buffer).

Data were processed using BIAevaluation 3.1 software using the 1:1 Langmuir model. The suitability of the fit was measured by the values of the Refractive Index (RI) which have to tend to zero and the κ² value.

The results are summarized in the following table 15

TABLE 15

| | Ab | | | | | | |
|---|---|---|---|---|---|---|---|
| | capture | Rmax | RI | | | | |
| | range | range | range | | kₒₙ | k_{off} | K_{D} |
| Antibodies | (RU) | (RU) | (RU) | κ² | (1/M · s) | (1/s) | (nM) |
| 1022C3 | 279.7 | 120 | 4.59 | 2.27 | 2.84 × | 3.60 × | 1.27 ± |
| | 269.6 | 98.1 | −4.57 | | 10⁵ ± | 10⁻⁴ ± | 0.05 |
| | | | | | 2.31 × | 12.3 × | |
| | | | | | 10³ | 10⁻⁶ | |

### EXAMPLE 5: BINDING EVALUATION AND FRET INHIBITION OF THE DEGLYCOSYLATED CDRH1

The 1022C3 posses an N-glycosylation site, that is post translationaly modified in the secreted protein, located in CDRH1. To determine the influence of the glycosylation site m1022C3 was enzymatically deglycosylated a process that converts the Asparagine residue to Aspartic acid.

The 1022C3 was deglycosylated using two glycosidases in a sequential manner. 1 μL of neuraminidase (New England Biolabs, P0720S, 50 000 U/mL) was added to 20 μg of a 1 mg/mL mAb solution and the mixture was incubated under gentle agitation at 37° C. overnight. 1 μL of Peptide-N-Glycosidase F (New England Biolabs, P0704S, 500 000 U/mL) was then added following by another incubation step overnight at 37° C.

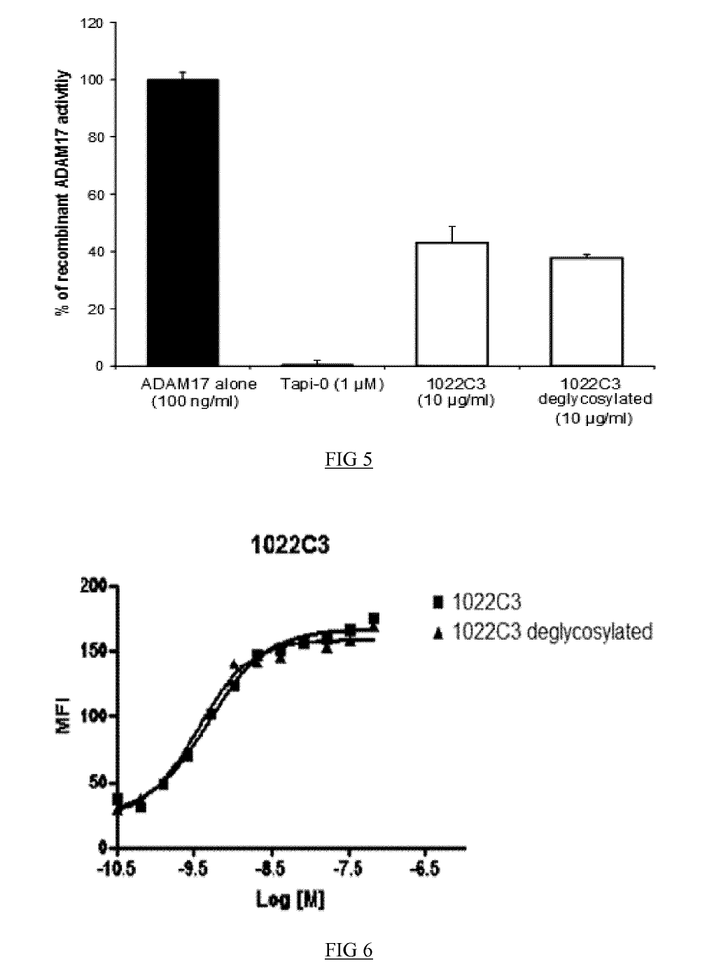

Enzymatic deglycosylation of 1022C3 did not reduce the inhibitory activity of 1022C3 in vitro evaluated in a FRET peptide cleavage assay (FIG. 5) retaining parental mAb levels of inhibition.

The enzymatically deglycosylated 1022C3 was evaluated for binding to the tumour cell line NCI-H1299 and was shown to have retained the binding capacity of the parental antibody (FIG. 6).

### EXAMPLE 6: SPECIFIC BINDING TO ADAM17

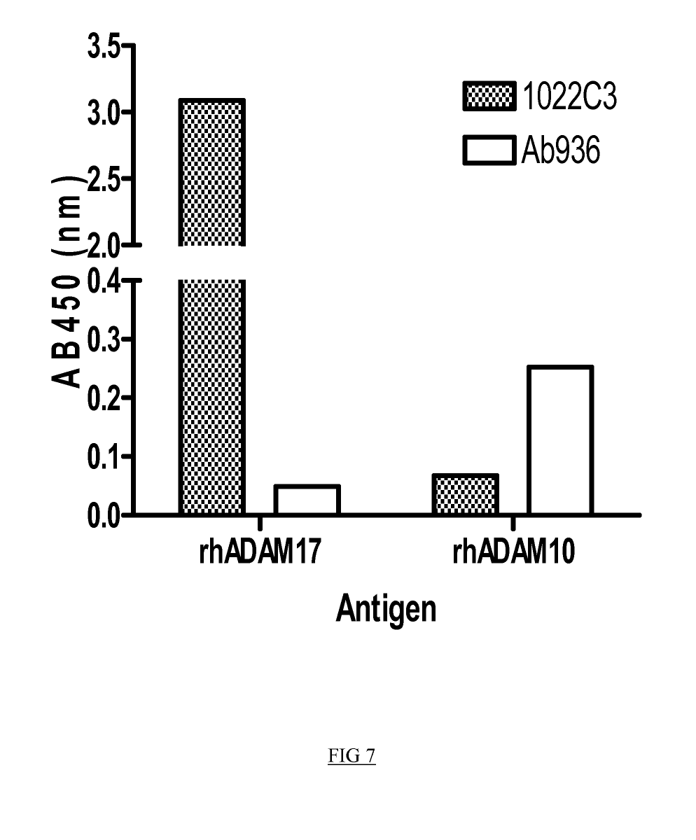

The binding of 1022C3 to human ADAM17 and ADAM10 was determined by ELISA in comparison to the anti human ADAM10 antibody AB936 (R&D Systems). A ninety six well ELISA plate was coated with 100 μl/well of recombinant human (rh)ADAM17 (930-ADB, R&D Systems) at a concentration of 1 μg/ml or rhADAM10 (AD936, R&D Systems) at a concentration of 2.5 μg/ml in PBS. The coating solution was incubated overnight at 4° C. The plates were then blocked with PBS containing 0.5% gelatin (#22151, Serva Electrophoresis GmbH, Heidelberg, Germany) for 2 h at 37° C. The saturation buffer discarded by flicking plates, 100 μl of 1022C3 at a concentration of 1 μg/ml in PBS or 5 μg/ml of anti human ADAM10 polyclonal (AB936, R&D Systems) was added to the ELISA plates and incubated for 1 h at 37° C. After three washes, 100 μl horseradish peroxidase-conjugated anti human (A7164, Sigma) or anti goat (115-035-164, Jackson ImmunoResearch Europe Ltd) antibody solution diluted 1/5000 in PBS were incubated for 1 h at 37° C. After three washes, 100 μl/well TMB substrate (#UP664782, Uptima, Interchim, France) was added. After a 10 min incubation time at room temperature, the reaction was stopped using 1 M sulphuric acid and the optical density at 450 nm was measured (FIG. 7).

### EXAMPLE 7: COMPARISON OF THE MURINE 1022C3 (m1022C3) WITH ITS HUMANIZED FORM (hz1022C3) ON THE CaOV3 XENOGRAFT MODEL

In order to compare the m1022C3 with its humanized form, the CaOV3 xenograft model was set up by cell engraftments on SCID mice as described above.

CaOV3, an ovarian carcinoma cell line, expressing ADAM17 (ABC=20 000), was selected for in vivo evaluations.

The person skilled in the art would easily determine the expression level of ADAM17 by any known technique such as cytometry, immunohistochemistry, Antibody Binding Capacity (ABC), etc. As a non limitative example, the expression level can be determined by measuring by cytometry the Antibody Binding Capacity (ABC) of a labelled antibody to ADAM17. In an embodiment, the tumour cell is considered as expressing ADAM17 with an ABC of at least 5000. In another embodiment, the tumour cell is considered as expressing ADAM17 with an ABC of at least 10000.

Mice were injected subcutaneously at D0 with 7×10⁶ cells. When tumours reached approximately 120 mm³ (19 days post tumour cell injection), animals were divided into two groups of 5 mice with comparable tumour size and treated intraperitoneally with a loading dose of 10 mg/kg and then weekly with maintenance doses of 5 mg/kg of m1022C3 and hz1022C3 monoclonal antibody or 2.5 mg/kg and then weekly with maintenance doses of 1.25 mg/kg. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

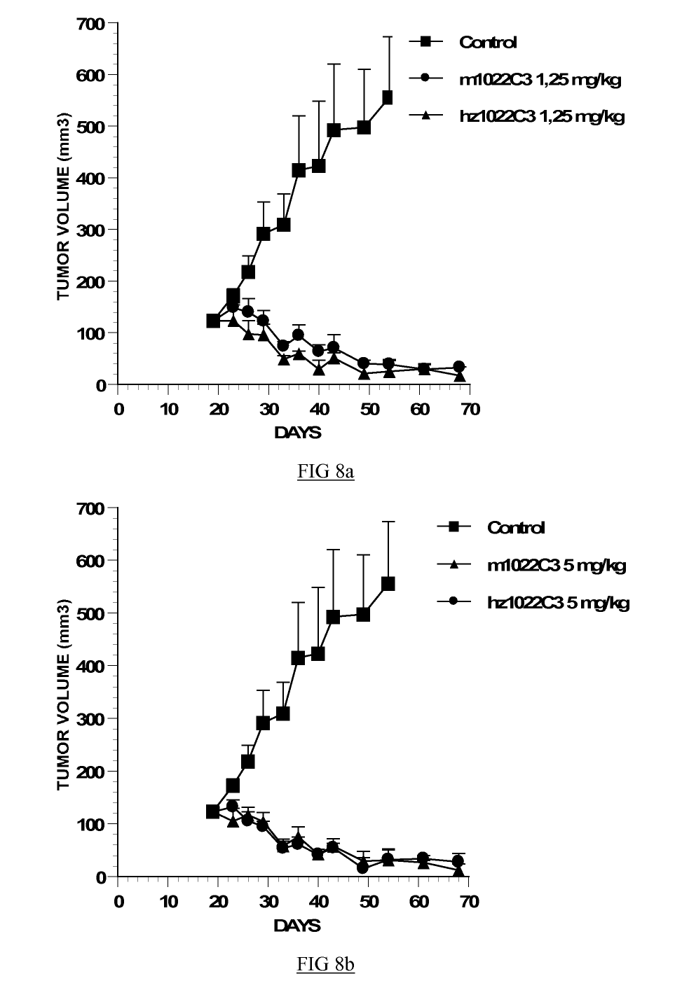

Results presented in FIGS. 8a and 8b demonstrated that the two compounds are comparable with tumour inhibitions reaching respectively 93% and 94% for m1022C3 and hz1022C3 when used at 1.25 mg/kg and 94% for both antibodies when used at 5 mg/kg.

### EXAMPLE 8: IN VIVO EVALUATION OF THE 1022C3 ANTIBODY

For all in vivo evaluations, six to eight weeks old athymic mice were used. They were housed in sterilized filter-topped cages, maintained in sterile conditions and manipulated according to French and European guidelines.

ADAM17, EGFR, HER2 expression levels were determined by staining, 1×10⁵ cells/100 μl in FACS buffer (PBS containing 1% BSA and 0.01% sodium azide) incubated for 20 min. at 4° C. with increasing concentrations of the MAB9301 (Clone 111633, R&D systems), 225 and 4D5 respectively in order to determine a saturating concentration. Cells were then washed three times in FACS buffer. Cells were resuspended and incubated for 20 min. at 4° C. with a goat anti-mouse IgG-Alexa 488 antibody (Invitrogen Corporation, Scotland, # A11017). Cells were then washed three times in FACS buffer. Labelled cells were then resuspended in 100 μl of FACS buffer prior to analysis with a Facscalibur cytometer (Becton Dickinson, Le Pont-de-Claix, France). Propidium iodide was added to analyse only viable cells. In parallel, QIFIKIT beads were used for the determination of antibody-binding and antigen density per cell by flow cytometry and monoclonal antibody binding. QIFIKIT contains a series of beads, 10 μm in diameter and coated with different, but well-defined quantities of mouse mAb molecules. The beads mimic cells with different antigen densities which have been labelled with a primary mouse mAb. The quantified antigen is expressed in Antibody-Binding Capacity (ABC) units.

8.1 A431 Xenograft Model (Wt): Established Tumours

A431, an epidermoid carcinoma cell line, expressing ADAM17 (ABC=17 000), was selected for in vivo evaluations. Mice were injected subcutaneously at D0 with 10×10⁶ cells. When tumours reached approximately 100 mm³ (25 days post tumour cell injection), animals were divided into 3 groups of 6 mice with comparable tumour size and treated intraperitoneally with a loading dose of 10 mg/kg and then weekly with maintenance doses of either 5 mg/kg of 1022C3 or 225 antibodies. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

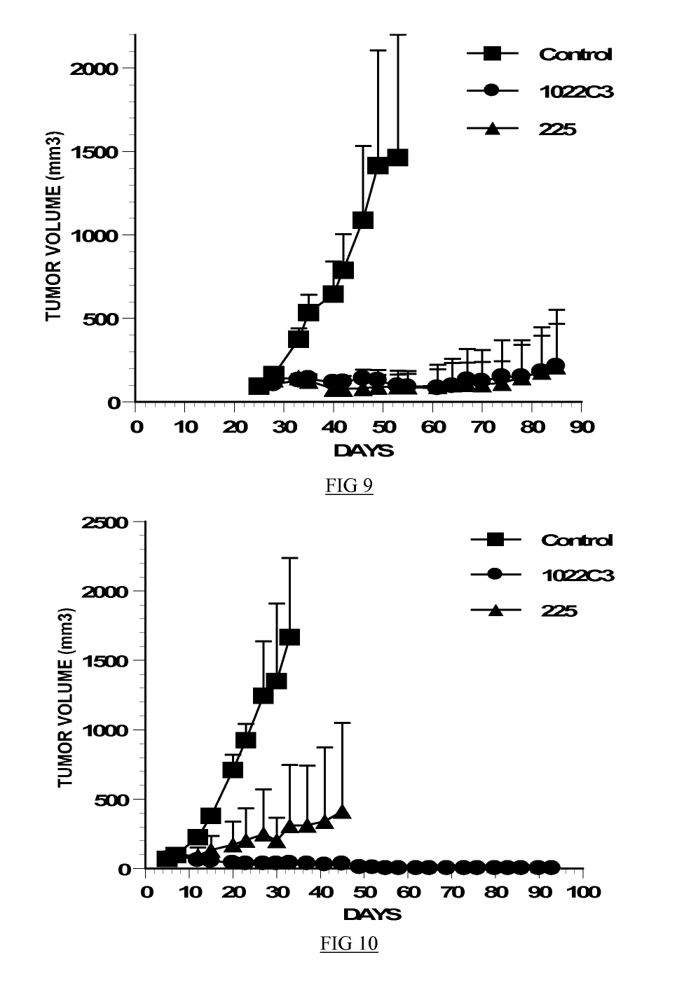

The results obtained were summarized in FIG. 9. They showed a dramatic tumour inhibition (94% at D53) mediated by both antibodies.

8.2 A431-AREG Xenograft Model: Established Tumours

A431-AREG was selected for in vivo evaluations. Mice were injected subcutaneously at D0 with 10×10⁶ cells. When tumours reached approximately 70 mm³ (5 days post tumour cell injection), animals were divided into groups of 6 mice with comparable tumour size and treated intraperitoneally with a loading dose of 10 mg/kg and then weekly with maintenance doses of either 5 mg/kg of 1022C3 or 225 antibodies (FIG. 10) or with a loading dose of 10 mg/kg and then weekly with maintenance doses of 5 mg/kg of 1022C3 or 1040H5 antibodies (FIG. 11). A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

The results obtained were summarized in FIGS. 10 and 11. They showed a dramatic tumour inhibition (98% at D33) mediated by the 1022C3 whereas 225 and 1040H5 showed a weaker activity: 82 and 87% of growth inhibition respectively. These last two antibodies do not induce growth regression as observed with 1022C3.

8.3 A431-HB-EGF Xenograft Model: Established Tumours

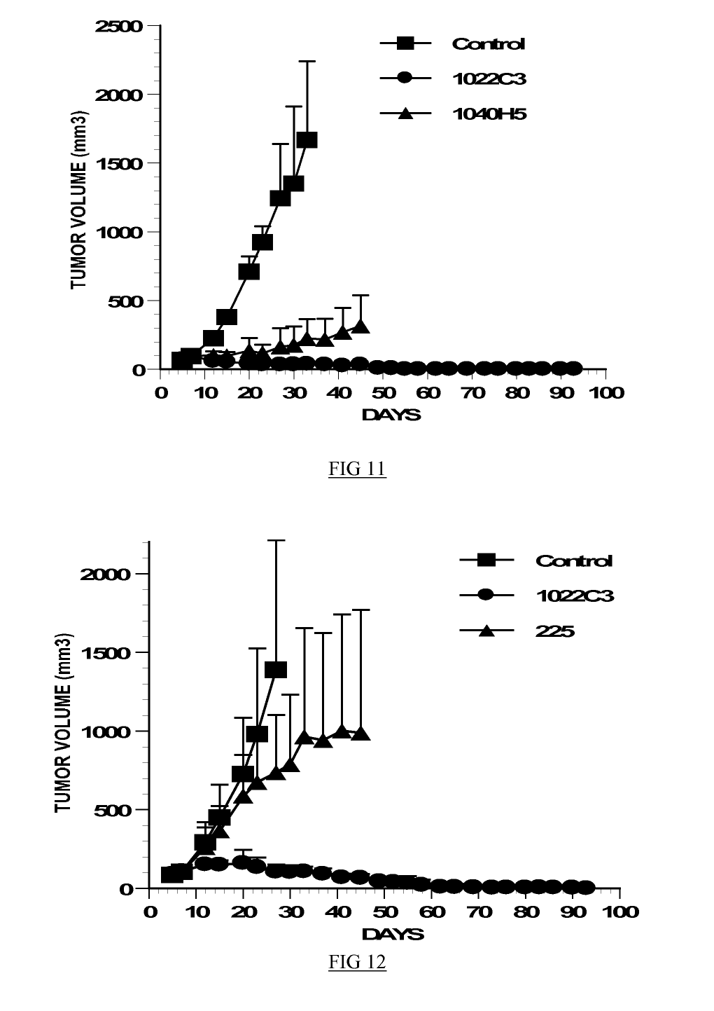

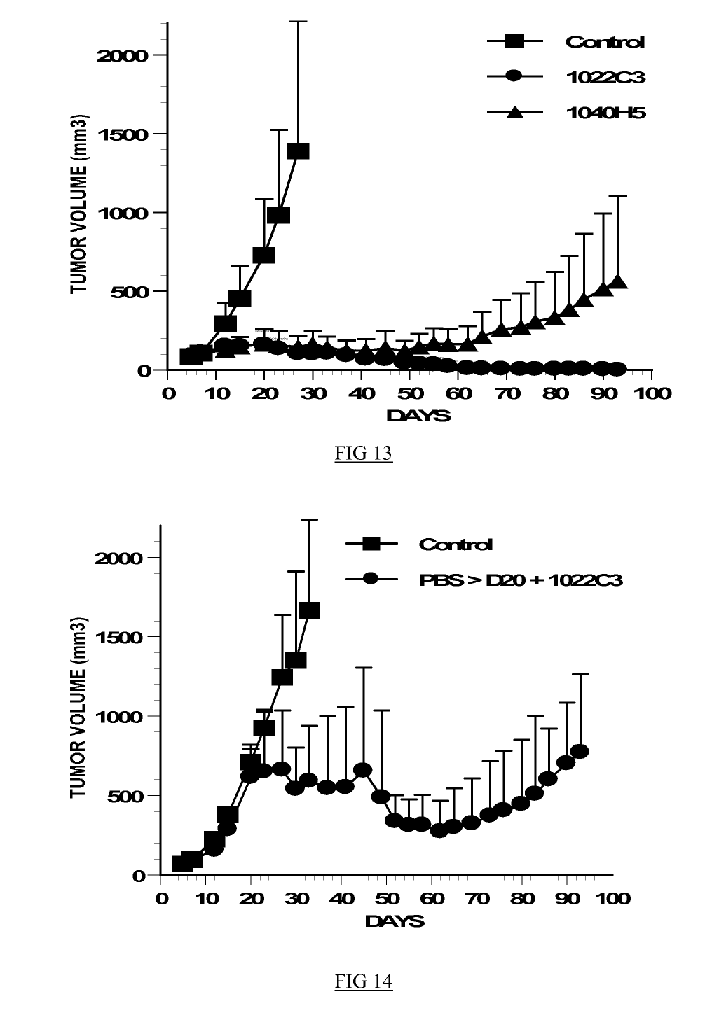

A431-HB-EGF was selected for in vivo evaluations. Mice were injected subcutaneously at D0 with 10×10⁶ cells. When tumours reached approximately 90 mm³ (5 days post tumour cell injection), animals were divided into groups of 6 mice with comparable tumour size and treated intraperitoneally with a loading dose of 10 mg/kg and then weekly with maintenance doses of either 5 mg/kg of 1022C3 or 225 antibodies (FIG. 12) or with a loading dose of 10 mg/kg and then weekly with maintenance doses of 5 mg/kg of the 1022C3 or 1040H5 antibodies (FIG. 13). A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

The results obtained were summarized in FIGS. 12 and 13. They showed a dramatic tumour inhibition (92% at D33) mediated by the 1022C3 whereas 225 looses its activity (FIG. 12) and 1040H5 showed weaker activity (FIG. 13): 47 and 90% of growth inhibition respectively. These last two antibodies do not induce growth regression as is observed with 1022C3.

8.4 1022C3 Robustness in A431-Substrate Models A431-AREG and A431-HB-EGF

To improve robustness of 1022C3 therapy, A431-HB-EGF and A431-AREG were selected for in vivo evaluation. Mice were injected subcutaneously at D0 with 1×10⁷ cells. When tumours reached approximately 700 mm³ (20 days post tumour cell injection), animals were divided into groups of 6 mice with comparable tumour size and treated intraperitoneally with a loading dose of 10 mg/kg and then weekly with maintenance doses of 5 mg/kg of 1022C3 (FIGS. 14 and 15). A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

The results obtained are summarized in FIGS. 14 and 15, and demonstrate a stabilization of tumour growth mediated by 1022C3 for both models.

8.5. Impact of 225 Therapy on 1022C3 Anti-Tumour Response

As high level of circulating ligands (HB-EGF or AREG) is not an exclusion factor for patient selection, we decided to explore the anti-tumoral response of 1022C3 after a EGFR targeted therapy using 225. A431-AREG and A431-HB-EGF were selected for these in vivo evaluations. Mice were injected subcutaneously at D0 with 1×10⁷ cells. When tumours reached approximately 70 to 90 mm³ (5 days post tumour cell injection for A431-AREG and A431-HB-EGF respectively), animals were divided into groups of 6 mice with comparable tumour size and treated intraperitoneally with a loading dose of 10 mg/kg and then weekly with maintenance doses of 5 mg/kg of 225 antibodies from D5 to D20. At D20, 225 therapy was replaced by 1022C3 therapy using the same injection protocol described above for 225. A control group received only the vehicle as previous experiments performed in this model demonstrated that no difference in tumour growth was observed between mice treated with vehicle and mice injected with an isotype control. The mice were followed for the observation of xenograft growth rate. Tumour volume was calculated by the formula: π/6×length×width×height.

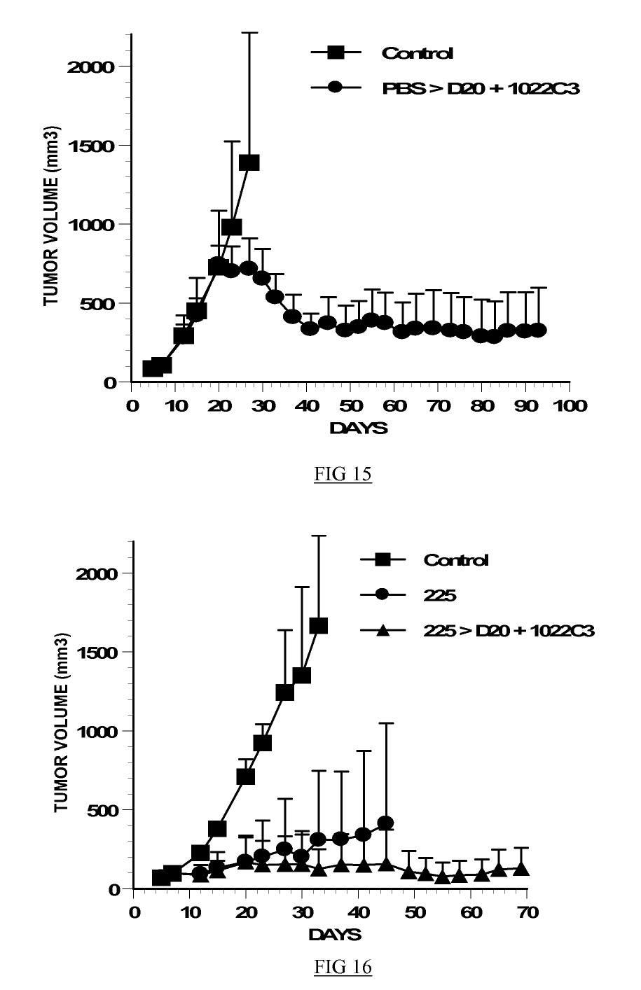

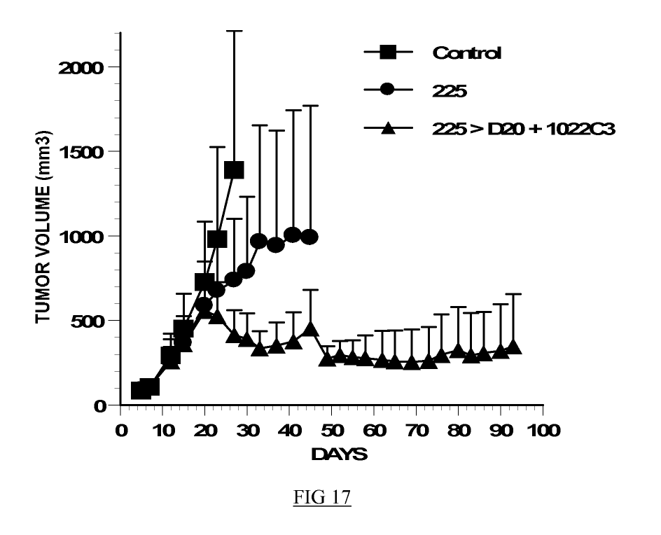

The results obtained are summarized in FIGS. 16 and 17 for the A431-AREG and A431-HB-EGF models respectively. The change from treatment with 225 to 1022C3 demonstrated enhanced anti tumoural effect in both models.

1.-19. (canceled)

20. A method of treating ADAM17 substrate dependent tumors, comprising administering to a patient in need thereof a pharmaceutical composition comprising an effective amount of an ADAM17 antibody, or an antigen-binding fragment thereof, said ADAM17 antibody having the following properties:
a) it binds to ADAM17 with a Kd of 3 nM or less;
b) it recognizes an epitope within the membrane proximal domain (MPD) of ADAM17, said MPD being localized between the residues 564 and 642;
c) it does not bind to ADAM10;
d) it inhibits the cellular shedding of at least one ADAM17 substrate with an IC₅₀ of 200 pM or less;
e) it has an off rate for ADAM17 of K_{off} of 3×10⁻⁴ or smaller;
f) it inhibits the growth and/or proliferation in vivo of at least one tumor cell expressing ADAM17;
g) it does not bind to the murine ADAM17; and
h) it binds to the cynomolgus ADAM17.

21. The method of claim 20, wherein said ADAM17 substrate dependent tumors are:
(i) tumors characterized by an elevated level of at least one ADAM17 substrate compared to the basal level of said at least one substrate, or
(ii) tumors that are resistant or refractory to treatment with an ErbB therapy.

22. The method of claim 20, wherein said ADAM17 antibody inhibits the shedding of at least one substrate selected from TNF-α, TGF-α, AREG, HB-EGF with an IC₅₀ of 500 pM or less.

23. The method of claim 20, wherein 7 said ADAM17 antibody inhibits the shedding of the substrates TNF-α, TGF-α, AREG and HB-EGF with an IC₅₀ of 500 pM or less.

24. The method of claim 20, wherein the said ADAM17 antibody, or an antigen-binding fragment thereof, comprises:
i) a heavy chain domain comprising CDR-H1, CDR-H2 and CDR-H3 of sequence SEQ ID No. 1, 2 and 3, respectively, and
ii) a light chain domain comprising CDR-L1, CDR-L2 and CDR-L3 of sequence SEQ ID No. 4, 5, and 6, respectively.

25. The method of claim 20, wherein said antibody is an affinity-matured mutant of the ADAM17 antibody of claim 24.

26. The method of claim 20, wherein said affinity-matured mutant antibody comprises a CDR-H1 of sequence SEQ ID No. 7 or SEQ ID No. 8.

27. The method of claim 20, wherein said affinity-matured mutant antibody comprises heavy chain variable domain of sequence SEQ ID No. 11 or SEQ ID No. 12.

28. A method of treating tumors that are refractory or resistant to treatment with an ErbB therapy, comprising administering to a patient in need thereof a pharmaceutical composition comprising an effective amount of an ADAM17 antibody, or an antigen-binding fragment thereof, said ADAM17 antibody having the following properties:
a) it binds to ADAM17 with a Kd of 3 nM or less;
b) it recognizes an epitope within the membrane proximal domain (MPD) of ADAM17 localized between the residues 564 and 642;
c) it does not bind to ADAM10;
d) it inhibits the cellular shedding of at least one ADAM17 substrate with an IC₅₀ of 200 pM or less;
e) it has an off rate for ADAM17 of K_{off} of 3×10⁻⁴ or smaller;
f) it inhibits the growth and/or proliferation in vivo of at least one tumor cell expressing ADAM17;
g) it does not bind to the murine ADAM17; and
h) it binds to the cynomolgus ADAM17.

29. The method of claim 28, wherein said tumors that are refractory or resistant to treatment with an ErbB therapy are:
(i) tumors with elevated levels of ErbB ligands compared to the level before the treatment with an ErbB therapy, or
(ii) tumors with elevated levels of ErbB ligands compared to healthy control.

30. The method of claim 28, wherein the ErbB therapy comprises administration of an EGFR antibody or an EGFR Kinase inhibitor, a Her2 antibody, or a Her2 kinase inhibitor, a Her3 antibody or a Her3 kinase inhibitor.

31. The method of claim 28, wherein said ADAM17 antibody inhibits the shedding of at least one substrate selected from TNF-α, TGF-α, AREG, HB-EGF with an IC₅₀ of 500 pM or less.

32. The method of claim 28, wherein 7 said ADAM17 antibody inhibits the shedding of the substrates TNF-α, TGF-α, AREG and HB-EGF with an IC₅₀ of 500 pM or less.

33. The method of claim 28, wherein the said ADAM17 antibody, or an antigen-binding fragment thereof, comprises:
i) a heavy chain domain comprising CDR-H1, CDR-H2 and CDR-H3 of sequence SEQ ID No. 1, 2 and 3, respectively, and
ii) a light chain domain comprising CDR-L1, CDR-L2 and CDR-L3 of sequence SEQ ID No. 4, 5, and 6, respectively.

34. The method of claim 28, wherein said antibody is an affinity-matured mutant of the ADAM17 antibody of claim 33.

35. The method of claim 28, wherein said affinity-matured mutant antibody comprises a CDR-H1 of sequence SEQ ID No. 7 or SEQ ID No. 8.

36. The method of claim 28, wherein said affinity-matured mutant antibody comprises heavy chain variable domain of sequence SEQ ID No. 11 or SEQ ID No. 12.

37. A method of treating ADAM17 substrate dependent tumors, comprising administering to a patient in need thereof an ADAM17 antibody named 1022C3, or an antigen-binding fragment thereof, said antibody comprising:
i) a heavy chain domain comprising CDR-H1, CDR-H2 and CDR-H3 of sequence SEQ ID No. 1, 2 and 3, respectively, and
ii) a light chain domain comprising CDR-L1, CDR-L2 and CDR-L3 of sequence SEQ ID No. 4, 5, and 6, respectively.

38. The method of claim 37, wherein said antibody is chimeric or humanized.

39. The method of claim 38, wherein said chimeric antibody comprise:
i) a heavy chain of a sequence selected in the group consisting of: SEQ ID No. 33 and SEQ ID No. 34; and/or
ii) a light chain of sequence SEQ ID No. 35.

40. The method of claim 38, wherein said humanized antibody comprise:
i) a heavy chain of a sequence selected in the group consisting of: SEQ ID No. 39, SEQ ID No. 40, SEQ ID No. 41, SEQ ID No. 42, SEQ ID No. 43, and SEQ ID No. 44; and/or
ii) a light chain of a sequence selected in the group consisting of: SEQ ID No. 45, SEQ ID No. 46, and SEQ ID No. 47.

41. The method of claim 40, wherein said antibody is an affinity-matured mutant of the ADAM17 antibody of claim 37.

42. The method of claim 41, wherein said affinity-matured mutant antibody comprises a CDR-H1 of sequence SEQ ID No. 7 or SEQ ID No. 8.

43. The method of claim 41, wherein said affinity-matured mutant antibody comprises a heavy chain variable domain of sequence SEQ ID No. 11 or SEQ ID No.

44. A method of inhibiting the growth of tumor cells that are refractory or resistant to ErbB therapy in a subject, said method comprising contacting said tumor cells with an effective amount of an ADAM17 antibody, or an antigen-binding fragment thereof, said ADAM17 antibody having the following properties:
a) it binds to ADAM17 with a Kd of 3 nM or less;
b) it recognizes an epitope within the membrane proximal domain (MPD) of ADAM17 localized between the residues 564 and 642;
c) it does not bind to ADAM10;
d) it inhibits the cellular shedding of at least one ADAM17 substrate with an IC₅₀ of 200 pM or less;
e) it has an off rate for ADAM17 of K_{off} of 3×10⁻⁴ or smaller;
f) it inhibits the growth and/or proliferation in vivo of at least one tumor cell expressing ADAM17;
g) it does not bind to the murine ADAM17; and
h) it binds to the cynomolgus ADAM17.

45. The method of claim 44, wherein said ADAM17 antibody inhibits the shedding of at least one substrate selected from TNF-α, TGF-α, AREG, HB-EGF with an IC₅₀ of 500 pM or less.

46. The method of claim 44, wherein the said ADAM17 antibody inhibits the shedding of the substrates TNF-α, TGF-α, AREG and HB-EGF with an IC₅₀ of 500 pM or less.

47. The method of claim 44, wherein said tumors that are refractory or resistant to treatment with an ErbB therapy are:
(i) tumors with elevated levels of ErbB ligands compared to the level before the treatment with an ErbB therapy, or
(ii) tumors with elevated levels of ErbB ligands compared to healthy control.

48. The method of claim 44, wherein said ErbB therapy comprises administration of an EGFR antibody or an EGFR Kinase inhibitor, a Her2 antibody, or a Her2 kinase inhibitor, a Her3 antibody, a Her3 kinase inhibitor.

49. A method of inhibiting the growth of tumor cells that are refractory or resistant to ErbB therapy in a subject, said method comprising contacting said tumor cells with an effective amount of an ADAM17 antibody, or an antigen-binding fragment thereof, said ADAM17 antibody comprising:
i) a heavy chain domain comprising CDR-H1, CDR-H2 and CDR-H3 of sequence SEQ ID No. 1, 2 and 3, respectively, and
ii) a light chain domain comprising CDR-L1, CDR-L2 and CDR-L3 of sequence SEQ ID No. 4, 5, and 6, respectively.

50. A method of inhibiting the growth of tumor cells that are refractory or resistant to ErbB therapy in a subject, said method comprising contacting said tumor cells with an effective amount of an affinity-matured mutant of an ADAM17 antibody, or an antigen-binding fragment thereof, said ADAM17 antibody comprising:
i) a heavy chain domain comprising CDR-H1, CDR-H2 and CDR-H3 of sequence SEQ ID No. 1, 2 and 3, respectively, and
ii) a light chain domain comprising CDR-L1, CDR-L2 and CDR-L3 of sequence SEQ ID No. 4, 5, and 6, respectively.

51. The method of claim 50, wherein said affinity-matured mutant antibody comprises a CDR-H1 of sequence SEQ ID No. 7 or SEQ ID No. 8.

52. The method of claim 51, wherein said affinity-matured mutant antibody comprises heavy chain variable domain of sequence SEQ ID No. 11 or SEQ ID No. 12.
